(19) United States
(12) Patent Application Publication
(10) Pub. No.: US 2022/0249763 A1
(43) Pub. Date: Aug. 11, 2022

(54) NEGATIVE PRESSURE WOUND DRESSING

(71) Applicant: AROA BIOSURGERY LIMITED, Auckland (NZ)

(72) Inventors: Dorrin ASEFI, Auckland (NZ); Alister Todd JOWSEY, Auckland (NZ); Isaac Tristram Tane MASON, Auckland (NZ); Brian Roderick WARD, Waiau Pa (NZ)

(73) Assignee: AROA BIOSURGERY LIMITED, Auckland (NZ)

(21) Appl. No.: 17/608,305

(22) PCT Filed: May 7, 2020
(86) PCT No.: PCT/NZ2020/050044

§ 371 Date: Nov. 2, 2021

Related U.S. Application Data
(60) Provisional application No. 62/844,690, filed on May 7, 2019.

(51) Int. Cl. A61M 1/00; A61L 15/40; A61L 15/42; A61L 15/64; A61L 15/26; A61F 13/02

(52) U.S. Cl. CPC A61M 1/915; A61L 15/40; A61L 15/425; A61L 15/64; A61M 1/92; A61F 13/0216; A61F 13/0263; A61M 1/985; A61L 15/26

(57) ABSTRACT

The invention relates to a multi-layer wound dressing for applying negative pressure to a wound. The dressing comprises a bioresorbable layer, for placement in contact with the wound; a fluid impermeable occlusive outer layer; a fluid porous porting layer positioned between the outer layer and the bioresorbable layer; and a fluid conduit in fluid communication with the porting layer, for coupling to a source of negative pressure. The porting layer comprises a multiplicity of fluid pathways between the conduit and the bioresorbable layer to allow for fluid transfer through the layers of the dressing to and from the bioresorbable. The bioresorbable layer comprises a plurality of apertures to enable exudate to flow from the wound to the porting layer.

### FIELD OF THE INVENTION

This invention relates to a wound dressing, in particular a dressing for the application of negative pressure and/or the instillation of treatment fluids to a wound.

### BACKGROUND

The technique of applying negative pressure to augment the healing of soft tissues has been utilised for many years with the core principle of the therapy remaining largely unchanged.

In the context of treating open wounds, negative pressure wound therapy (NPWT) typically involves the placement of porous materials such as an open-cell foam, reticulated foam or gauze onto the wound site, sealing the wound cavity with an occlusive layer and applying a negative pressure to the sealed wound environment (see FIGS. 1 and 2). The clinical efficacy of this treatment is well supported in areas such as for acute and chronic wounds, which have demonstrated accelerated formation of granulation tissue in open wounds in response to the treatment.

While the open architecture of the porous wound contacting layer allows for the effective application of pressure to the wound and removal of wound exudate, a shortcoming with present NPWT dressing constructs is the susceptibility for healing granulation tissue to grow in to the porous wound contacting layer. This results in trauma to the newly formed tissue when the foam layer is removed. To prevent or minimise tissue in-growth it is necessary to regularly change the dressing which requires additional time and expense. Furthermore, the repeated dressing changes can induce acute trauma to the periwound or intact skin area around the wound further compounding the overall treatment time.

Collagen scaffolds, extracellular matrices and tissue graft materials provide another helpful means to promote tissue growth and the regeneration of tissue in wounds. These bioresorbable collagen-based materials contain biophysical and biochemical elements which support the regenerating tissue through the various stages of healing. The collagen material properties within these scaffolds can vary greatly which is largely attributed to the varying xenogeneic or allogeneic origins and differing processing methods used during manufacture.

Collagen is a resorbable structural protein with a high affinity for water and so collagen scaffolds can draw water into the fine pores of the material. Therefore, the use of these materials is primary limited to the treatment of wounds with low levels of wound exudate. The retained fluid within the scaffold can prevent cellular migration and proliferation which can inhibit effective incorporation into the wound. In the context of NPWT, these materials present a substantial barrier to the passage of negative pressure to the wound interface, with the associated pressure drop rendering the NPWT treatment ineffective.

It is an object of at least preferred embodiments of the present invention to address one of the abovementioned disadvantages and/or to at least provide the public with a useful alternative.

In this specification where reference has been made to patent specifications, other external documents, or other sources of information, this is generally to provide a context for discussing features of the invention. Unless specifically stated otherwise, reference to such external documents or sources of information is not to be construed as an admission that such documents or such sources of information, in any jurisdiction, are prior art or form part of the common general knowledge in the art.

### SUMMARY OF THE INVENTION

In a first aspect, the invention broadly consists in a wound dressing for applying negative pressure to a wound, the dressing comprising: a bioresorbable layer, for placement in contact with the wound; a liquid impermeable occlusive outer layer; a fluid porous porting layer positioned between the outer layer and the bioresorbable layer; and a fluid conduit in fluid communication with the porting layer, for coupling to a source of negative pressure; wherein the porting layer defines a multiplicity of fluid pathways between the conduit and the bioresorbable layer; and wherein the bioresorbable layer comprises a plurality of apertures or slits to enable fluid flow from the wound to the porting layer.

The bioresorbable layer may comprise a plurality of mechanically interlocked bioresorbable sheets. The bioresorbable layer may have a first sheet having a plurality of lugs and a second sheet having a plurality of apertures, each lug of the first sheet being located through a respective aperture in the second sheet to interlock the first sheet with the second sheet.

In an embodiment, the bioresorbable sheets comprise extracellular matrix (ECM). The ECM may comprise reticulum.

In an embodiment, the bioresorbable layer comprises a plurality of apertures, the apertures defining fluid pathways.

In an embodiment, the apertures comprise two intersecting slots to form a cross shape and to define one or more flaps in the bioresorbable layer, wherein the flaps are movable to increase the size of the opening provided by each aperture. The slots may be substantially X-shaped, Y-shaped, C-shaped, U-shaped, or V-shaped.

In an embodiment, the apertures are formed through the bioresorbable layer by removing a slug of material from the bioresorbable layer.

In an embodiment, the bioresorbable layer comprises a plurality of slits defining the fluid pathways, each slit defining one or more flaps in the bioresorbable layer, wherein the flaps are movable to increase the size of the opening provided by the slit. The slits may be substantially X-shaped, Y-shaped, C-shaped, U-shaped, or V-shaped.

In an embodiment, the slits or slots are die cut from the bioresorbable layer.

The slits or slots preferably define flaps that allow the aperture to open under pressure.

In an embodiment, the porting layer is compliant and porous. For example, the porting later may comprise a fluid-permeable foam such as PVA (Polyvinyl alcohol) foam.

In an embodiment, wherein an upper surface of the porting layer is undulating.

The porting layer may comprise an antimicrobial treatment.

In an embodiment, the dressing further comprises a pressure distribution layer between the porting layer and the occlusive layer. The pressure distribution layer may comprise an open cell foam or a three-dimensional fabric.

In an embodiment, the pressure distribution layer comprises a plurality of fluid flow channels that are substantially perpendicular to the interface between the foam layer and the pressure distribution layer to allow fluid to flow through the pressure distribution layer.

In an embodiment, the conduit comprises a distal end portion having an opening in fluid communication with the porting layer.

In an embodiment, the conduit distal end portion is substantially arch-shaped.

In some forms, the conduit comprises a dual lumen conduit comprising a strut positioned along a central axis of one of the lumens to prevent the conduit from collapsing under compression.

Optionally, the conduit comprises a lumen which is elliptical in shape.

In an embodiment, the conduit is a dual lumen conduit comprising a primary conduit to apply a negative pressure to the dressing and a secondary conduit for introducing fluid to the dressing or for facilitating pressure measurement.

In an embodiment, the dressing further comprises a sleeve comprising a port for receiving a portion of the conduit therein in a secure arrangement to attach the conduit to the dressing. The sleeve may comprise an elastomeric material.

In some embodiments, the sleeve forms a divider between a negative pressure receiving area of the dressing and an ambient pressure area.

In an embodiment, the occlusive layer comprises a substantially transparent region and the porting layer comprises one or more viewing apertures beneath the transparent region to enable visual inspection of at least a portion of the bioresorbable layer.

In an embodiment, the occlusive layer comprises a polyurethane sheet having an adhesive surface.

In an embodiment, the wound dressing comprises a mouldable adhesive seal for surrounding a wound, wherein the seal comprises butyl rubber, a filler, and a tackifying resin. Preferably, the seal is removable and re-sealable against a patient's skin. In some forms, the seal is non-curing. In some embodiments, the seal is removable from a skin surface by stretching the adhered seal.

In a second aspect, the invention broadly consists in a mouldable and removable adhesive seal for surrounding a wound, the seal comprising butyl rubber, a filler, and a tackifying resin.

In an embodiment, the seal is repositionable and deformable.

In an embodiment, the seal is non-curing.

In an embodiment, the seal is removable from a skin surface by elongating the adhered seal.

In a third aspect, the invention broadly consists in an adhesive seal application system comprising the mouldable adhesive seal described in relation to the second aspect, and further comprising a first removable release sheet adhered to one side of the adhesive seal, and a second removable release sheet adhered to a second side of the adhesive seal, wherein the second removable release sheet is stretchable.

In an embodiment, the second removable release sheet comprises silicone. Optionally, a removable protector sheet adhered to the second removable release sheet.

In some forms, the first removable release sheet is paper based and comprises an adhesive contacting side coated in silicone.

In some forms, the adhesive seal is elongate and stretchable.

Optionally, the seal is non-curing.

In a fourth aspect, the invention broadly consists in a wound treatment system comprising a wound dressing as described above in relation to the first aspect, and the mouldable adhesive seal described above in relation to the second aspect, wherein the mouldable adhesive seal is applied around the perimeter of the wound to the patient's skin.

In an embodiment, the occlusive layer is adhered over the mouldable adhesive seal.

In an embodiment, a negative pressure source is coupled to the conduit to apply a negative pressure to the wound.

In an embodiment, the system comprises a reservoir for collecting exudate removed from the dressing.

This invention may also be said broadly to consist in the parts, elements and features referred to or indicated in the specification of the application, individually or collectively, and any or all combinations of any two or more said parts, elements or features. Where specific integers are mentioned herein which have known equivalents in the art to which this invention relates, such known equivalents are deemed to be incorporated herein as if individually described.

The term ‘comprising’ as used in this specification and claims means ‘consisting at least in part of’. When interpreting statements in this specification and claims that include the term ‘comprising’, other features besides those prefaced by this term can also be present. Related terms such as ‘comprise’ and ‘comprised’ are to be interpreted in a similar manner.

It is intended that reference to a range of numbers disclosed herein (for example, 1 to 10) also incorporates reference to all rational numbers within that range and any range of rational numbers within that range (for example, 1 to 6, 1.5 to 5.5 and 3.1 to 10). Therefore, all sub-ranges of all ranges expressly disclosed herein are hereby expressly disclosed.

As used herein the term ‘(s)’ following a noun means the plural and/or singular form of that noun. As used herein the term ‘and/or’ means ‘and’ or ‘or’, or where the context allows, both.

### BRIEF DESCRIPTION OF THE DRAWINGS

The present invention will now be described by way of example only and with reference to the accompanying drawings.

FIG. 1 is a perspective view of a prior art negative pressure wound dressing.

FIG. 2 is a perspective view of a further prior art negative pressure wound dressing.

FIG. 3 is a cut-away perspective view of a first embodiment negative pressure wound dressing described herein.

FIG. 4 is an exploded perspective view of the wound dressing of FIG. 3.

FIG. 5 is a cut-away perspective view of a second embodiment negative pressure wound dressing.

FIGS. 6(i) and 6(ii) are perspective views of the porting layer of FIG. 5, where FIG. 6(i) shows the upper surface of the layer, and FIG. 6(ii) shows the underside.

FIGS. 7(i) to 7(iii) are detail views showing a cut away portion of the porting layer of FIG. 5, where FIG. 7(i) is an isometric view showing the upper surface of the layer, FIG. 7(ii) is an overhead perspective view, and FIG. 7(iii) is an underside perspective view.

FIGS. 8(i) and 8(ii) are perspective views of an alternative embodiment porting layer having undulating ribs, where FIG. 8(i) shows the upper surface of the layer, and FIG. 8(ii) shows the underside.

FIGS. 9(i) to 9(iii) are detail views showing a cut away portion of the porting layer of FIGS. 8(i) and 8(ii), where FIG. 9(i) is an isometric view showing the upper surface of the layer, FIG. 9(ii) is an overhead perspective view, and FIG. 9(iii) is an underside perspective view.

FIGS. 10(i) and 10(ii) are perspective views of an alternative embodiment porting layer having square ribs, where FIG. 10(i) shows the upper surface of the layer, and FIG. 8(ii) shows the underside.

FIGS. 11(i) to 11(iii) are detail views showing a cut away portion of the porting layer of FIGS. 10(i) and 10(ii), where FIG. 11(i) is an isometric view showing the upper surface of the layer, FIG. 11(ii) is an overhead perspective view, and FIG. 1(iii) is an underside perspective view.

FIGS. 12(i) to 12(iv) are a series of schematics showing the block build′ process of regenerating tissue forming in passages of the porting layer and the atraumatic removal following successful treatment FIG. 13 is a cut-away perspective view showing a portion of an exemplary multi-sheet lugged bioresorbable layer according to one embodiment.

FIGS. 14(i) and 14(ii) are partial section views of exemplary multi-sheet lugged bioresorbable layers, where FIG. 14(i) shows an embodiment having lugs formed from the top sheet engaging the underlying sheets, and FIG. 14(ii) shows an embodiment additionally having a bottom lugged sheet engaging the overlying sheets.

FIGS. 15(i) and 15(ii) illustrate an embodiment of the bioresorbable layer having X-shaped apertures, where FIG. 15(i) shows the layer flat, and FIG. 15(ii) shows the edges of the apertures deforming to allow more flow through the apertures.

FIGS. 16(i) to 16(iii) further illustrate the operation of the bioresorbable layer apertures of FIGS. 15(i) and 15(ii), where FIG. 16(i) is a plan view of an aperture on a flat sheet, FIG. 16(ii) illustrates the aperture deforming when the sheet is placed on a contoured wound surface, and FIG. 16(iii) shows the edges of the apertures deformed to allow more flow through the aperture.

FIGS. 17(i) to 17(iii) illustrate fluid flow paths through the bioresorbable layer of FIGS. 16(i) to 16(iii), where FIG. 17(i) is a cut-away perspective view illustrating the sheet placed on a contoured wound surface; FIG. 17(ii) is a schematic side view showing the edges of the apertures deformed to allow more flow through the aperture, and FIG. 17(iii) illustrates fluid flow paths through the apertures.

FIG. 18 illustrates a prior art graft with staggered linear expansion fenestrations, where FIG. 18(i) is a cut-away perspective view illustrating the graft placed on a contoured wound surface; FIG. 18(ii) illustrates deformation of the fenestrations to accommodate wound surface undulations, and FIG. 18(iii) illustrates the restricted flow through the graft.

FIGS. 19(i) to 19(iv) are perspective views illustrating four exemplary alternative embodiment bioresorbable layers, where FIG. 19(i) shows an embodiment having oval apertures, FIG. 19(ii) shows an embodiment having X-shaped slits, FIG. 19(iii) shows an embodiment having Y-shaped slits, and FIG. 19(iv) shows an embodiment having C-shaped slits.

FIGS. 20(i) and 20(ii) are plan views illustrating two further exemplary alternative embodiment bioresorbable layers, where FIG. 20(i) shows an embodiment having circular apertures, and FIG. 20(ii) shows an embodiment having C-shaped slots.

FIGS. 21(i) and 21(ii) illustrate the operation of the bioresorbable layer apertures of FIG. 19(iv), where FIG. 21(i) is a perspective view showing deformation of the bioresorbable layer to conform to a contoured wound surface, and FIG. 21 (ii) shows the flaps, defined by the slits, deformed to allow more flow through the apertures.

FIGS. 22(i) and 22(ii) illustrate the operation of the bioresorbable layer apertures of FIG. 19(iii), where FIG. 22(i) is a perspective view showing deformation of the bioresorbable layer to conform to a contoured wound surface, and FIG. 22(ii) shows the flaps, defined by the slits, deformed to allow more flow through the apertures.

FIGS. 23(i) and 23(ii) illustrate the operation of the bioresorbable layer apertures of FIG. 19(i), where FIG. 23(i) is a perspective view showing deformation of the bioresorbable layer to conform to a contoured wound surface, and FIG. 23(ii) shows flow through the apertures.

FIGS. 24(i) to 24(iii) illustrate an alternative form bioresorbable layer formed from reticulum, where FIG. 24(i) is a perspective view showing the textured top surface of a reticulum sheet, FIG. 24(ii) is a perspective view of the top of the layer, with X-shaped slots, and FIG. 24(iii) is a perspective view of the underside of the layer.

FIG. 25 is an illustrative perspective view showing the wound dressing described herein place over a foot wound.

FIG. 26 is view corresponding to FIG. 25, showing the process of removing the mouldable strip of the dressing, from the foot.

FIG. 27 is an illustrative perspective view showing placement of the wound dressing described herein on an arm wound.

FIG. 28 is view corresponding to FIG. 27 showing the process of removing the mouldable strip of the dressing, from the arm.

FIG. 29 is a cross-sectional schematic showing the mouldable rubber adhered to skin.

FIG. 30 is a view corresponding to FIG. 29 showing removal of the mouldable rubber.

FIG. 31 is a cut-away perspective view of a negative pressure wound dressing according to a third embodiment, having a pressure distribution layer.

FIG. 32 is an exploded perspective view of the wound dressing of FIG. 31.

FIG. 33 is a cut-away perspective view of a negative pressure wound dressing according to a fourth embodiment, having an alternative embodiment pressure distribution layer.

FIG. 34 is a cross-sectional view of an embodiment dual lumen conduit, comprising a primary conduit to apply a negative pressure to the dressing and a secondary conduit for instilling fluid to the dressing or for pressure measurement.

FIGS. 35(i) and 35(ii) are illustrate views of an embodiment of a sleeve, where FIG. 35(i) relates to a view along the axis of the through-hole of the component, and FIG. 35(ii) relates to an isometric view of the sleeve.

FIGS. 36(i) and 36 (ii) are illustrative views of the embodiment dual lumen conduit of FIG. 34 assembled to the elastomeric sleeve component of FIGS. 35 (i) & 35 (ii), where the dual lumen conduit has been cut along the second end of the conduit to expose the primary and secondary conduits of the dual lumen conduit along a length.

FIG. 37 is a cut-away perspective view of a negative pressure wound dressing according to a fifth embodiment, incorporating the assembled dual lumen conduit and elastomeric sleeve component of FIGS. 36(i) and 36 (ii).

FIG. 38 is an exploded perspective view of the wound dressing of FIG. 37.

FIG. 39 is an exploded perspective view of an apparatus used to prepare a bioresorbable layer of the invention.

FIG. 40 is an exploded perspective view of the tooling used within apparatus described in FIG. 39.

FIGS. 41 (i) and 41 (ii) are cross-sectional views illustrating the process of the operating the apparatus in FIG. 39 to prepare a bioresorbable layer of the invention.

FIG. 42 is an exploded perspective view of the pressure drop measurement apparatus described herein.

FIG. 43 is chart displaying the results from measuring the pressure drop across various wound contacting devices in response to two different levels of applied negative pressure.

### DETAILED DESCRIPTION

### I. Definitions

The term “extracellular matrix” (ECM) as used herein refers to animal or human tissue that has been decellularised and provides a matrix for structural integrity and a framework for carrying other materials.

The term “decellularised” as used herein refers to the removal of cells and their related debris from a portion of a tissue or organ, for example, from ECM.

The term “polymeric material” as used herein refers to large molecules or macromolecules comprising many repeated subunits, and may be natural materials including, but not limited to, polypeptides and proteins (e.g. collagen), polysaccharides (e.g. alginate) and other biopolymers such as glycoproteins, or may be synthetic materials including, but not limited to, polypropylene, polytetrafluoroethylene, polyglycolic acid, polylactic acid, and polyester.

The term “interlock” or “interlocking” as used herein refers to the mechanical engagement of two or more overlapping sheets of material.

The term “sheet” as used herein refers to a substantially flat flexible section of ECM or polymeric material.

The term “lug” as used herein refers to a section of a sheet that has been partially cut out so that the lug remains fixedly attached to the sheet via a connection bridge.

In this specification and claims, the terms ‘negative pressure’ and ‘vacuum pressure’ may be used interchangeable to mean a gauge pressure less than an ambient pressure and an absolute pressure less than atmospheric pressure. Alternative terms include ‘sub-atmospheric pressure’, ‘suction pressure’ or ‘reduced pressure’. For example, a negative pressure or vacuum pressure of 100 mmHg is −100 mmHg gauge pressure or around 660 mmHg absolute pressure. The terms ‘higher’, ‘increase’, when used in relation to negative or vacuum pressure, are intended to mean higher or increasing negative pressure. For example, a gauge pressure of −150 mmHg (610 mmHg absolute) is higher than a gauge pressure of −100 mmHg (660 mmHg absolute). Similarly, in relation to the terms ‘lower’, ‘decrease’, when used in relation to negative or vacuum pressure, are intended to mean lower or decreasing negative pressure. For example, a gauge pressure of −100 mmHg is lower than a gauge pressure of −150 mmHg.

In this specification and claims, unless the context indicates otherwise, the term ‘exudate’ is intended to mean any fluid removed from a wound site of a patient. For example, exudate may comprise fluid produced by the patient, and/or fluid applied to the wound site by the system, including air or treatment fluid such as saline, or fluid providing medication, or fluid from a surgical intervention that may have introduced or administered treatment fluids to the wound site via a separate route such as by injection.

### II. Device

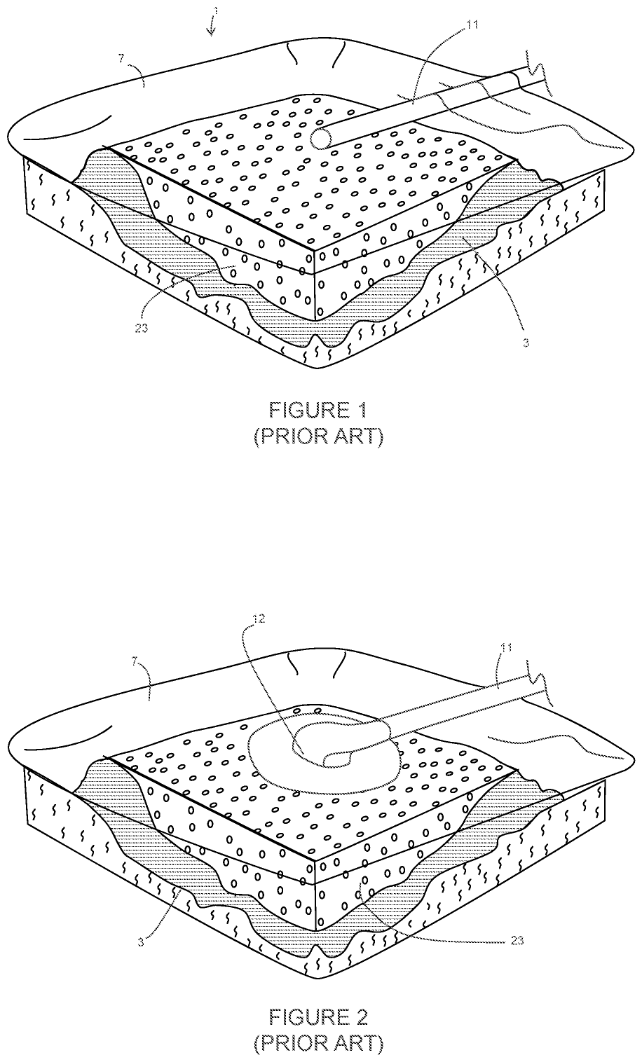

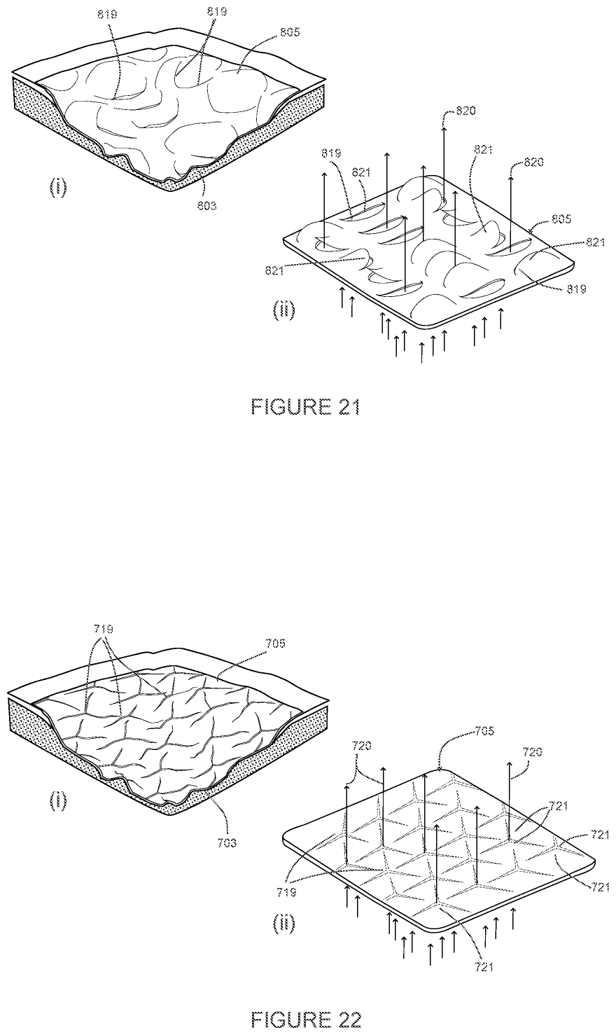

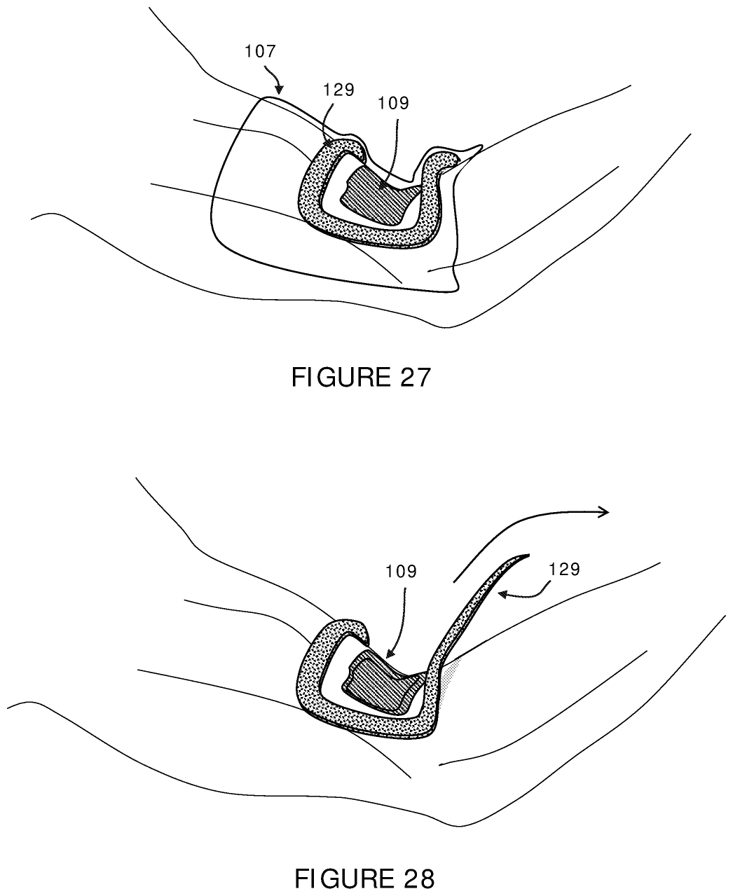

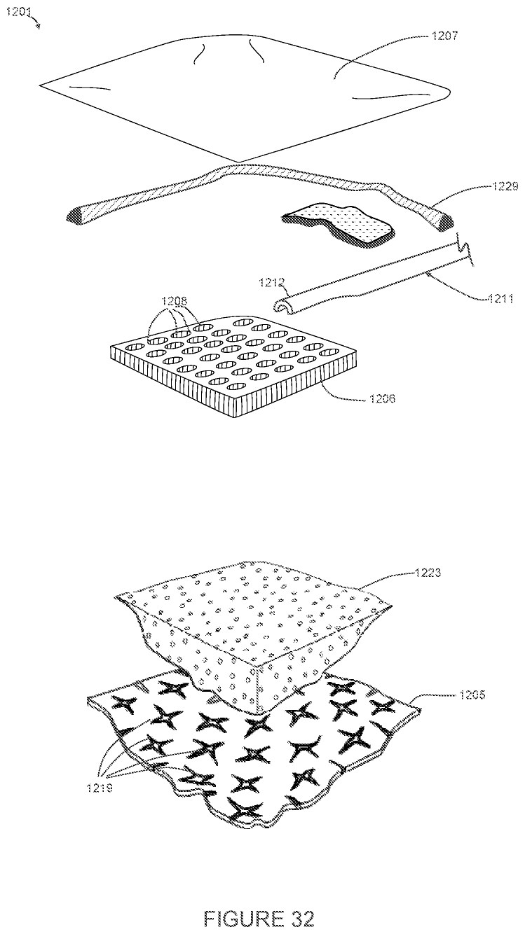

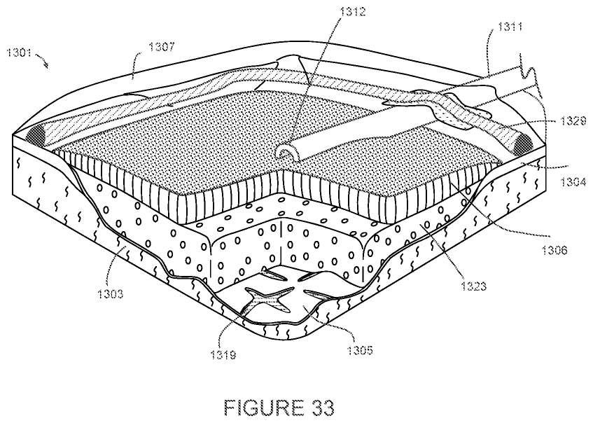

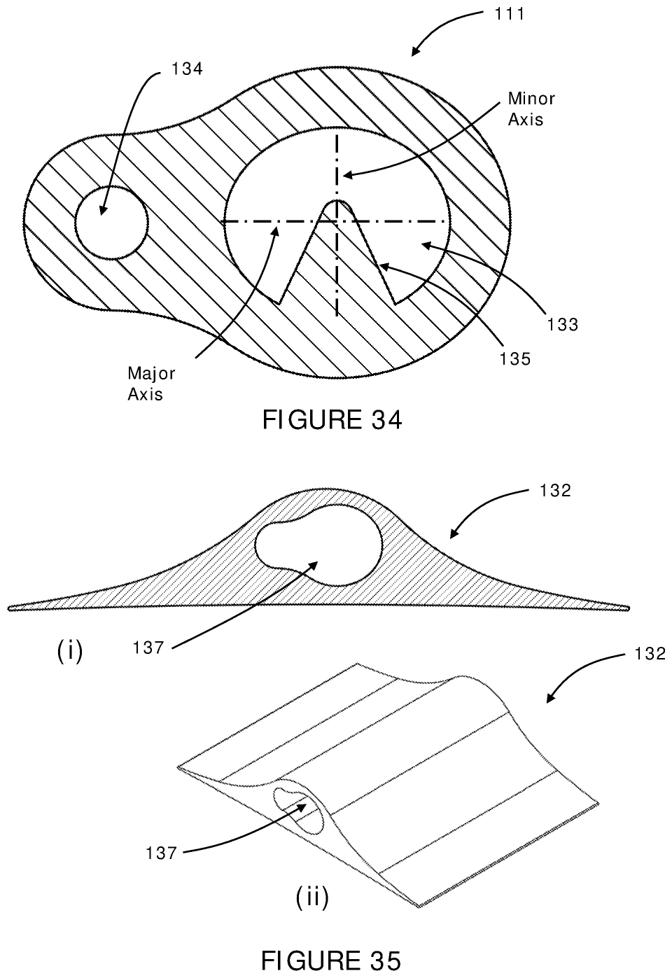

Various embodiments will now be described with reference to FIGS. 1 to 40. In these figures, like reference numbers are used in different embodiments to indicate like features, with the addition of a multiple of 100. Directional terminology such as the terms ‘front’, ‘rear’, ‘upper’, ‘lower’, and other related terms are used in the following description for ease of description and reference only, it is not intended to be limiting.

The invention generally provides a multi-layered wound dressing system that includes a wound dressing comprising at least one layer of bioresorbable material and at least one other layer of material comprising apertures that act as fluid ports to allow fluid to pass through the layers to and from the wound site. The fluid may be a gas or liquid or both. The multi-layer wound dressing also comprises an adhesive portion, such as a mouldable seal, that surrounds the wound site to define the boundary of a wound treatment region. Typically, the adhesive portion is provided on the intact skin outside the boundary of the wound. The adhesive portion additionally seals to a liquid impermeable occlusive layer to provide an enclosed environment around the wound and define the wound treatment region. The multi-layer wound dressing system also includes a negative pressure component that comprises at least one conduit having a distal end that terminates within the enclosed environment creating by the wound dressing and that allows treatment fluid to be delivered to the wound site and exudate to be removed from the wound site. The conduit also allows for negative pressure to be applied to the enclosed environment surrounding the wound. The negative pressure helps assist healing of the wound and therefore can reduce the time taken for the wound to heal.

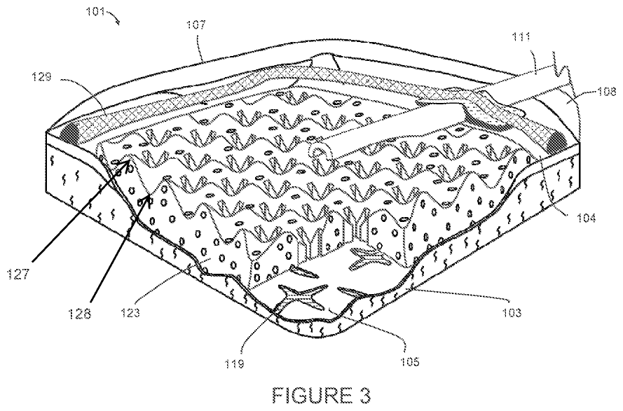

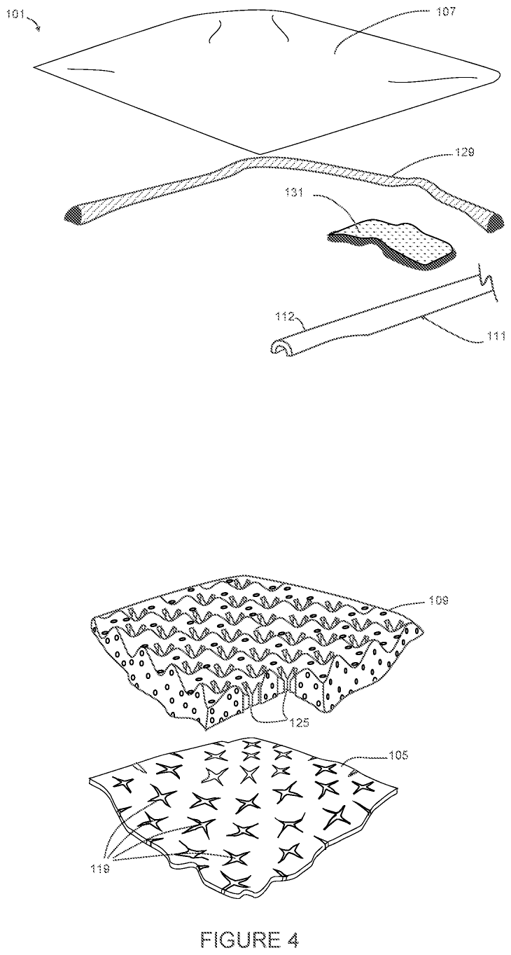

FIGS. 3 and 4 illustrate a first exemplary embodiment wound dressing 101 suitable for applying negative pressure to a wound 103. The dressing 101 comprises a bioresorbable layer 105 for placing in contact with a wound surface 103, a liquid impermeable occlusive outer layer 107, a porting layer 109 between the outer layer 107 and the bioresorbable layer 105, and a fluid conduit 111 in fluid communication with the porting layer 109, for coupling to a source of negative pressure.

Bioresorbable Layer

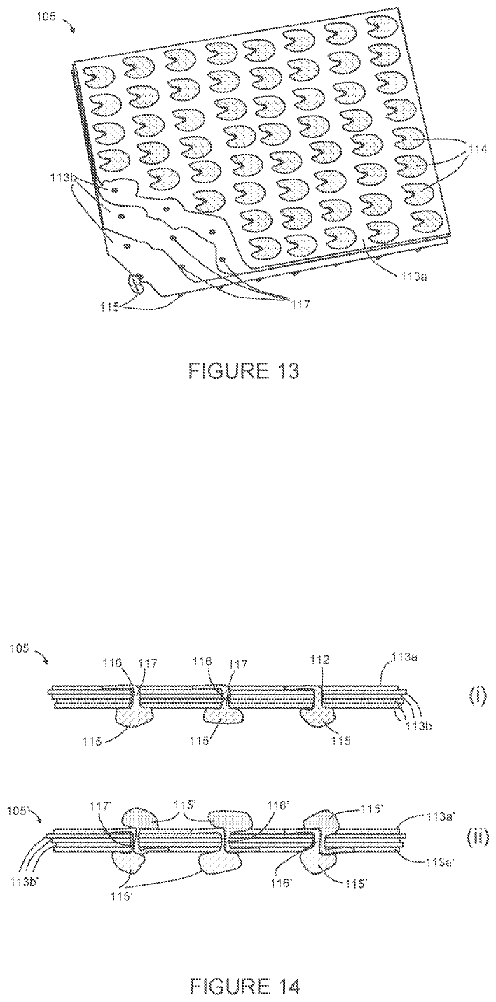

With reference to FIGS. 13 and 14, the wound contacting bioresorbable layer 105 comprises a flexible multi-sheet structure. In the embodiments exemplified herein, the bioresorbable layer 105 comprises a plurality of overlaid sheets 113a, 113b that are mechanically interlocked to each other, for example utilising portions of one or more of the sheets to engage with one or more of the other sheets. Mechanically interlocking the sheets 113a, 113b secures the sheets together without the need for the addition of other materials such as adhesives or sutures, or the need for treatments such as compression and dehydration. In addition, these multi-sheet structures have a greater combined tensile strength than individual sheets.

The multi-layer structure of the bioresorbable layer 105 may be produced according to the method described PCT application PCT/NZ2015/050215, which is incorporated herein by reference. Example multi-sheet interlocked bioresorbable layers 105, 105′ produced according to this method are illustrated in FIGS. 13, 14(i), and 14(ii)). In the embodiments shown, the bioresorbable layer 105 comprises a first, lugged sheet 113a, 113a having a plurality of lugs 115 formed by cutting a U-shaped or C-shaped slit in the first sheet to create a tab-like ‘lug’. The underlying or overlying sheets 113b have a plurality of perforations 117 and each lug 115 is pushed through the respective underlying or overlying perforations 117 to interlock the sheets together to create a lugged laminate. The resulting structure contains recesses 114 in the lug sheet where each lug 115 was cut from the sheet. Each lug 115, remains attached to its respective lug sheet 113, via a connection bridge 116, thereby interlocking the sheets to hold them together.

In the exemplary embodiments 105, 105′ shown in FIGS. 14(i) and (ii), there are three or four perforated sheets, but alternatively the bioresorbable layer may comprise more or fewer perforated sheets. The number of sheets superimposed at and secured at different points of the bioresorbable layer may vary, for example, if different properties are required at different areas of the bioresorbable layer.

In some embodiments, the bioresorbable layer may comprise more than one lug sheet 113a, for example, having both upper and lower lugged sheets as illustrated in FIG. 14(ii). In that embodiment, three perforated sheets 113b′ are sandwiched between a top lug sheet and a bottom lug sheet 113a′. Lugs 115′ from the top lug sheet have been pushed through the perforations 117′ of the middle sheets 113b′ to the underside of the lower lug sheet, and lugs 115′ from the lower lug sheet have been pushed through the perforations of the middle sheets 113b′ to the top surface of the top lug sheet.

The lugs of the lower lug sheet may be aligned with the lugs in the top sheet as shown in the embodiment of FIG. 14(ii), or they may be offset to prevent lugs on different lug sheets being pushed through the same perforations 117′. The mechanical properties of the product can be further tailored to the application needs by modifying the shape of the lugs, or utilising a different lugging pattern, size, density and/or shape.

The lugs 115, 115′ may or may not be pushed through all of the underlying or overlying sheets and, for embodiments with more than one lug sheet, may or may not be pushed through the other lug sheet.

The perforations 117, 117′ for the lugs in the multi-sheet lugged bioresorbable layer provide a plurality of micro-channels through the sheet. These channels advantageously assist with fluid flow from the wound through the bioresorbable layer and assist with pressure application to the wound due to the channels provided by the perforations for the lugs.

The bioresorbable layer sheets 113 comprise extracellular matrix (ECM) or a polymeric material. ECM-derived matrices for use in embodiments of the present invention are collagen-based biodegradable matrices comprising highly conserved collagens, glycoproteins, proteoglycans and glycosaminoglycans in their natural configuration and natural concentration. One extracellular collagenous matrix for use in this invention is ECM of a warm-blooded vertebrate. ECM can be obtained from various sources, for example, gastrointestinal tissue harvested from animals raised for meat production, including pigs, cattle and sheep or other warm blooded vertebrates. Vertebrate ECM is a plentiful by-product of commercial meat production operations and is thus a low cost tissue graft material. One exemplary method of preparing ECM is described in U.S. Pat. No. 8,415,159.

In some embodiments of the invention, resorbable polymeric material may be included in the bioresorbable layer as either lug sheets, pierced sheets, and/or in another three-dimensional form. For example, meshes comprising synthetic materials such as polyglycolic acid, polylactic acid and poliglecaprone-25 are will provide additional strength in the short-term, but will resorb in the long term. Alternatively, the polymeric material may be a natural material, or derived from a natural material, such as proteins (e.g. collagen), polysaccharides (e.g. alginate), glycoproteins or other materials.

In some embodiments, the bioresorbable layer 105 may comprise one or more sheets of reticulum 1113 (see FIG. 24) which may be produced according to the method described in PCT application PCT/NZ2009/000152, which is incorporated herein by reference. Reticulum is a propria-submucosa of the forestomach of a ruminant that possesses a unique raised ‘honeycomb’ appearance on the luminal surface of the tissue. These honeycomb features are created by a series of continuous native ridges comprised of predominately dense collagen which create an undulating and varied textured surface on the luminal face of the reticulum tissue. The abluminal surface is generally smooth in appearance following the delamination and removal of the muscle layer. While these raised ridges retain an element of elasticity, they are relatively incompressible when subjected to the negative pressure applied within wound therapy.

The raised ridges of the reticulum also assist with the distribution of pressure across the surface of each individual honeycomb pocket by preventing the collapse and sealing of the adjacent dressing materials, which is a unique feature of the native material. The bioresorbable layer 105 may additionally be treated for the delivery of bioactive materials to the wound site. The bioactive materials may be endogenous to the ECM used in the preparation of a graft product or may be materials that are incorporated into the ECM and/or polymeric material layers during or after manufacturing. Bioactive materials delivered to the wound site in this way are known to be beneficial for promoting cellular function, including wound healing and other desirable physiological and pharmacological functions.

In other embodiments, the bioresorbable layer 105 may comprise one or more sheets of ECM sourced from the rumen, which is another propria-submucosa of the forestomach of a ruminant and is also described within PCT application PCT/NZ2009/000152.

With reference to FIGS. 13, 14 and 24 (i) to 24 (iii) the use of forestomach tissue in the construction of a lugged multi-sheet bioresorbable layer 105 carries other additional benefits. As described within PCT application PCT/NZ2009/000152, tissue scaffolds sourced from forestomach tissue typically have a contoured luminal surface of varying appearance according to the particular origin of the scaffold (such as the rumen, reticulum or omasum) where the abluminal surface is generally smooth in appearance following the delamination and removal of the muscle layer. With particular reference to the rumen, the luminal surface of this tissue scaffold is populated with a number of surface protrusions known as papillae, which visually appear as small ‘hair-like’ features that project from the luminal surface. When a bioresorbable layer 105 is constructed according to the aforementioned lugged laminate process using rumen tissue, the resultant lugged laminate comprises an interstitial space formed in-between adjacent sheets of the laminate as a result of the papillae located within each interstitial space that prevent the adjacent sheets from forming a tight seal between the layers. This interstitial space is not limited to only laminates using rumen tissue. Other embodiments that comprise resorbable foams and other resorbable polymeric materials may also comprise an interstitial space between adjacent sheet layers.

The multi-sheet bioresorbable layer 105 comprises a plurality of primary apertures, which may comprise slits (formed from cuts made without removing material from the layer), slots (having spaced apart side edges as a result of removed material from the layer) or any other suitable form of opening such a regular or irregularly shaped opening, through the bioresorbable layer 105 to define a multiplicity of fluid pathways. These pathways enable fluid flow from the wound to the porting layer 109.

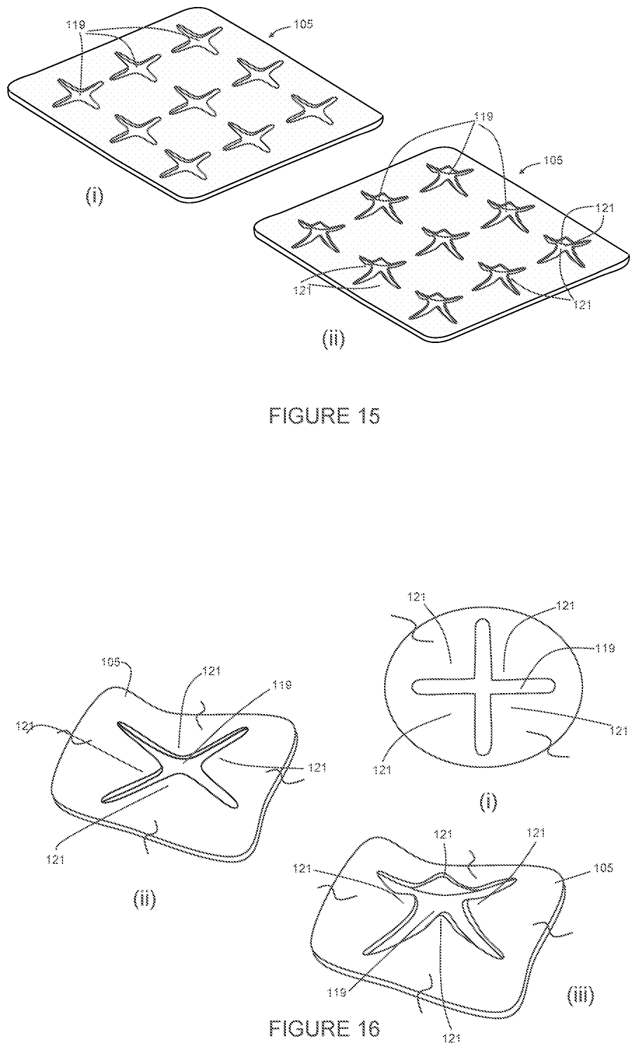

In one embodiment shown in FIGS. 15(i) to 17(iii), the apertures in the bioresorbable layer 105 comprise an array of x-shaped apertures formed by two intersecting slots 119. The bioresorbable layer is flexible, such that each X-shaped aperture may define four generally triangular flaps 121 in the bioresorbable layer. The two free edges of each triangular flap 121 are formed by a pair of slots that extend across the same generally central point in a perpendicular arrangement to each other to form a cross, with the third edge of the triangular flap forming a hinge with the body of the bioresorbable layer.

These x-shaped slots allow the bioresorbable layer 105 to flex to conform to undulations in a wound surface 103, as illustrated in FIG. 17(i), with the edges of a given slot able to move closer together to accommodate a concavity, or able to spread apart to accommodate a convex surface. Therefore, the bioresorbable layer is able to be substantially in full contact with the wound surface. The x-shaped apertures also provide other benefits by allowing the passage of wound exudate fluid through the layer and also allow the supplied negative pressure therapy to the wound surface 103 over a large equivalent area without removing a large area of material, thereby reducing the amount of bioactive material being delivered to the wound by the bioresorbable layer 105.

In another embodiment shown in FIG. 16(i) the x-shape formed by the intersecting slots 119 has a width and height of approximately 5.5 mm. The slot preferably has a width of approximately 0.5 mm, with approximately 5 mm² of resultant bioresorbable material removed from the layer 105. In contrast to this, if the apertures formed in the bioresorbable later are in the form of a circular perforation similar to that of FIG. 20 (i) with a diameter of approximately 5.5 mm then approximately 24 mm² of bioresorbable material must be removed to provide an aperture of this size and a comparable level of supplied negative pressure to the wound surface and capacity of fluid exchange through the bioresorbable layer 105 as that experienced using the cross shaped apertures of FIG. 16(i).

In other embodiments, the width and height of the x-shaped aperture may be longer in one direction than the other or may comprise a variety of different sized slots across the bioresorbable layer 105. The length of the slots forming the x-shape may range from about 3 mm to about 15 mm in width and length, with the width of each slot ranging from about 0.2 mm up to about 2 mm in width.

In some forms, apertures are provided in the bioresorbable layer in a generally regular arrangement, such as by being located in substantially aligned columns and rows. In other forms, the apertures may be arranged in an offset or staggered arrangement. In yet other forms, the apertures may be provided in an irregular arrangement or a random arrangement on the bioresorbable layer.

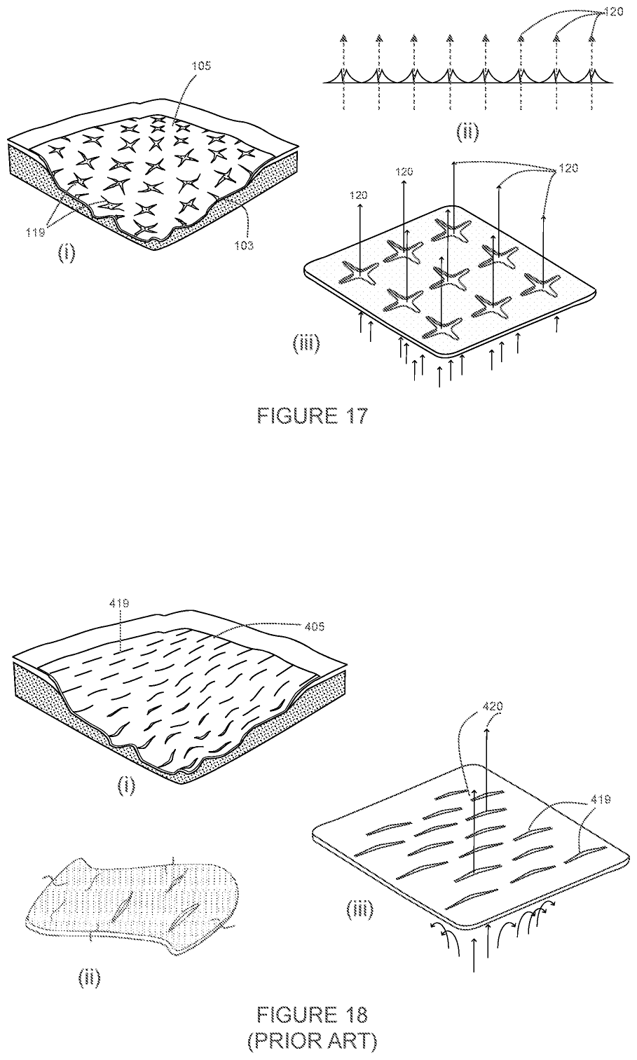

FIGS. 18(i) to 18(iii) show a known graft product which includes linear fenestrations/slits 419 rather than apertures formed as a result of material being removed from the product. If used in a dressing, these fenestrations/slits are less able to take up the undulations in both the longitudinal and transverse directions. The linear slits only provide a narrow opening for the fluid flow 420 through the graft as they are formed without removing material from the graft product. Consequently, the linear slits are only capable of allowing removal of fluid from a smaller region of the wound site than the apertures of the present invention. In addition, the linear fenestrations 419 close easily once the graft 405 is in situ, due to moisture taken up by the graft 405 causing swelling of the graft near the slits 419, which further closes the narrow openings. Regenerating tissue, wound fluids such as blood, red blood cells, fibrin and other wound phenomena, such as slough and healing tissue debris, also collect and populate in these narrow fenestrations and can cause the fenestrations to block. Furthermore, the lateral movement of the graft within the wound can cause the linear slit fenestrations to close with the slightest movement, preventing any fluid exchange through the layer.

In contrast, as illustrated in FIGS. 15(ii), 16(iii), 17(ii) and 17 (iii), the flaps 121 defined by the X-shaped apertures/slots of the dressing of the present invention are able to move upwards to increase the size of the opening and thereby to increase the size of the fluid pathways 120 provided by the slots 119. This may happen under pressure, for example due to the negative pressure being applied to the dressing, and allows fluid paths to be maintained even if there is some swelling of the material surrounding the slots. As well as facilitating the removal of fluid from the wound site, maintaining the fluid pathways allows a more efficient application of negative pressure to the wound site because larger openings in the bioresorbable layer are advantageously associated with less pressure drop over the thickness of the bioresorbable layer 105. Additionally, the X-shaped slots are also less prone to closing when the graft moves within the wound as the X-shaped openings skew in response to lateral movement to maintain the openings through the bioresorbable layer.

In alternative embodiments, the bioresorbable layer primary apertures 119 may have alternative shapes. For example, rather than being X-shaped, the apertures may be slots of another two dimensional shape such that they each define one or more flaps that is movable to accommodate contours and is movable to increase the size of the fluid pathway through the aperture. For example, suitable slot shapes include slots having a curved portion or comprising two or more linear portions arranged in an angle to form an arrowhead type arrangement. Each flap is created by two or more adjacent linear slots edges, or by a convex/curved slot, with the slot edge or edges defining the free (moving) edges of the flap and a virtual line between two distal ends of the curved slot or pair of slots forming the hinge of the flap. In some forms, rather than being X-shaped, in alternative embodiments the slots 119 may be Y-shaped, C-shaped, U-shaped, or V-shaped. Each Y-shaped slot defines three flaps, each C-shaped, U-shaped, or V-shaped slot defines one flap.

Each slot or other primary aperture is formed by removing material from the bioresorbable layer. For example, by blanking, by making spaced apart cuts and removing the intervening material, or by die cutting or laser cutting whereby material is removed by a single pass of the laser beam, the slot width corresponding to the laser beam width.

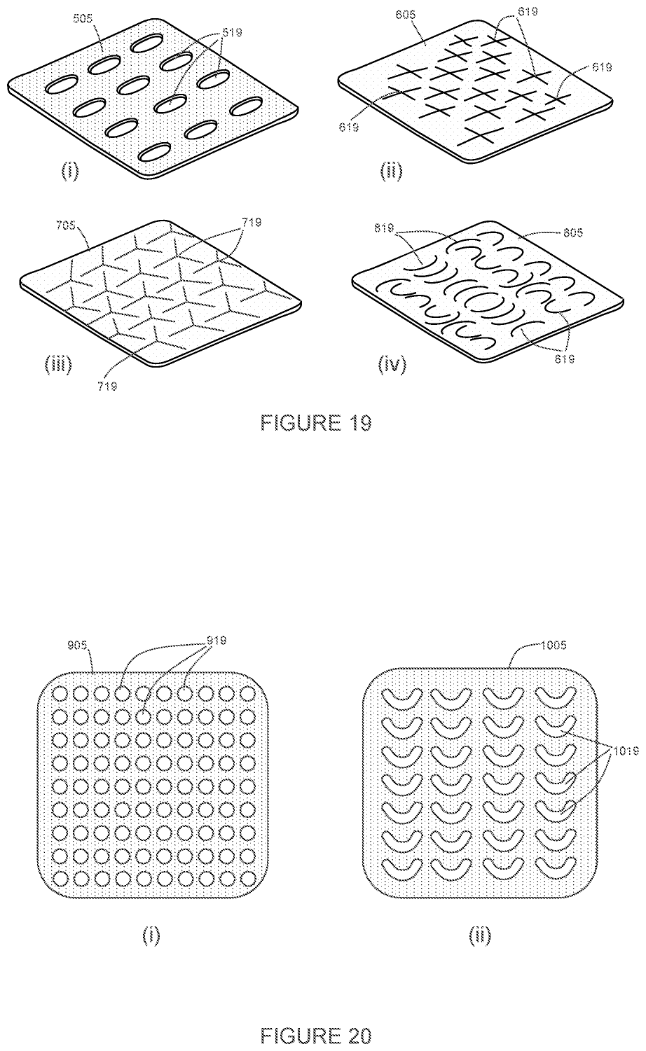

Alternatively, rather than a slot where material has been removed from the bioresorbable layer, the flaps for the fluid passages may be formed using shaped slits, created by cut lines where no material is removed from the sheet. The slits may be linear or of a curved shape as described above in relation to the slots, having a two-dimensional shape, or the slots may be formed by a non-linear cut or from interesting/irregular shaped cuts, such that each slit defines one or more flaps that is movable to accommodate contours of the wound site. The flaps are movable to ‘open’ to create a fluid pathway or increase the size of the fluid pathway through the layer at the slit. FIGS. 19(ii) to 19(iv) illustrate some exemplary slit arrangements. FIG. 19(ii) illustrates an embodiment 605 having X-shaped slits 619, with each slit 619 defining four flaps that are movable in the manner described above in relation to the X-shaped slots. FIGS. 19(iii), 22(i), and 22(ii) illustrate an embodiment 705 with Y-shaped slits 719, each slit being formed from three intersecting linear cuts to define three flaps 721. FIGS. 19(iv), 21(i), and 21(ii) illustrate an embodiment 805 with variously oriented C-shaped slits 819, with each slit 819 defining a single flap 821.

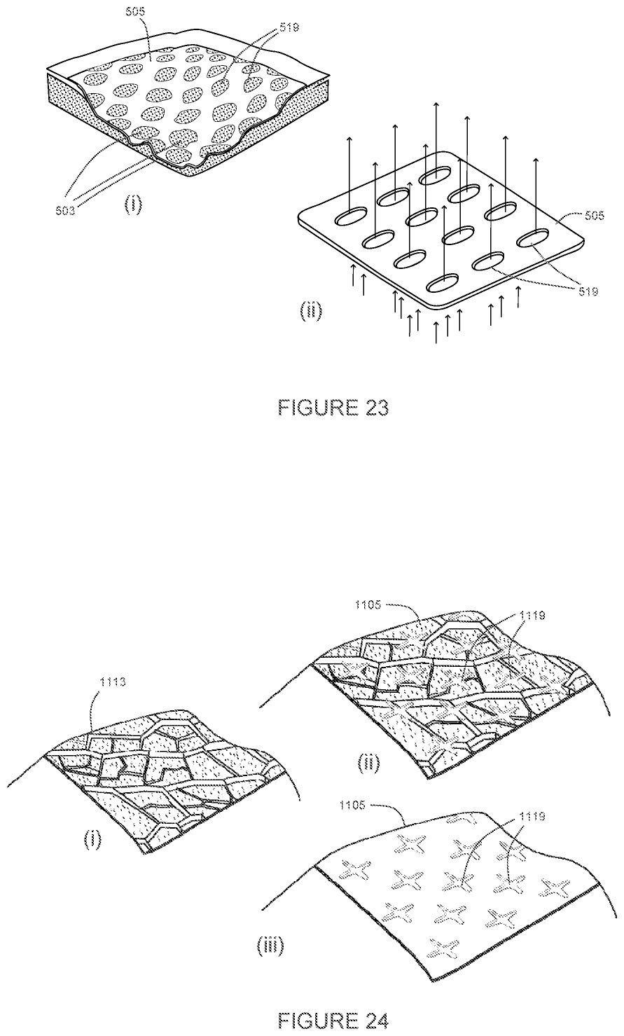

As a further alternative, the primary apertures in the bioresorbable layer may comprise a plurality of openings that don't form flaps, for example round apertures 919 as shown in FIG. 20(i), oval or oblong apertures 519 as shown in FIGS. 19(i), 23(i), and 23(ii), or other shaped apertures. In these embodiments, the bioresorbable layers do not comprise movable flaps to increase the size of the fluid pathway through the layer. Instead, the apertures provide a larger opening than the slots described above, for the efficient transfer of pressure to the wound and passage of fluid. However, this increase in the opening size is associated with a reduction of the area of the bioresorbable layer 505, 905 that is in contact with the wound surface, thereby reducing the therapy area. Sheets having larger apertures may also be more difficult to handle.

In some embodiments, to provide a lugged multi-layered dressing with apertures 119 within the bioresorbable layer 105 that do not interfere with the interlocking lugs, the apertures may be arranged in a grid pattern and the lugs 115 may be arranged between at least some of the adjacent apertures in a layer. The width of the apertures 119 may also be longer in one axis than the other to provide sufficient space on the layer for the interlocking lugs 115, such that the width of each primary aperture 119 may span the equivalent length of two or several adjacent lugs 115 but be limited in height. In other embodiments the apertures 119 within the bioresorbable layer 105 may be arranged in a staggered pattern.

Porting Layer

The porting layer 109 is positioned on top of the bioresorbable layer 105, between the bioresorbable layer and the occlusive layer 107. The porting layer 109 defines a multiplicity of fluid pathways between the conduit 111 and the bioresorbable layer 105, to enable the porting of pressure to the bioresorbable layer and for the passage of wound exudate out of the bioresorbable layer 105.

The porting layer 109 maintains spacing between the bioresorbable layer 105 and the occlusive outer layer 107 under the application of negative pressure to the dressing via the conduit 111. This porting layer 109 also provides some protection to the wound 103 by cushioning the wound, and assists to distribute the negative pressure from the conduit 111 to the bioresorbable layer across a wide area, rather than to the area immediately adjacent the conduit.

The porting layer 109 comprises a material selected to minimise the pressure drop across the layer 109 while also discouraging tissue-in growth. The porting layer 109 material must also provide sufficient structural integrity to allow for the passage of fluid through the porting layer under elevated levels of pressure, such as between approximately 125 mmHg to approximately 250 mmHg of vacuum pressure. In one embodiment, the porting layer comprises a compliant porous material such as a solid-state, water permeable synthetic foam. The foam is of at least a semi-open cell nature to allow fluid passage through the foam layer. The greater the porosity of the foam layer and the stiffer the material, the less the pressure drop.

In an embodiment, the porting layer 109 comprises an antimicrobial open cell foam or a semi-open/semi-closed cell foam such as a PVA foam. The foam is flexible and compressible to conform to and cushion the wound site 103. Some openness is required in the sub-layer 123 to allow the porting of pressure and the transfer of liquid through the layer. However, more open foams such as reticulated polyurethane are generally more susceptible to tissue in-growth, which is undesirable. Utilising a semi-closed foam such as PVA foam immediately adjacent to the bioresorbable layer reduces the ability for tissue to grow into the foam layer. Additionally, the porting layer 109 may also comprise a series of channels 125 to further improve the porting of pressure and the transfer of liquid through the porting layer 109.

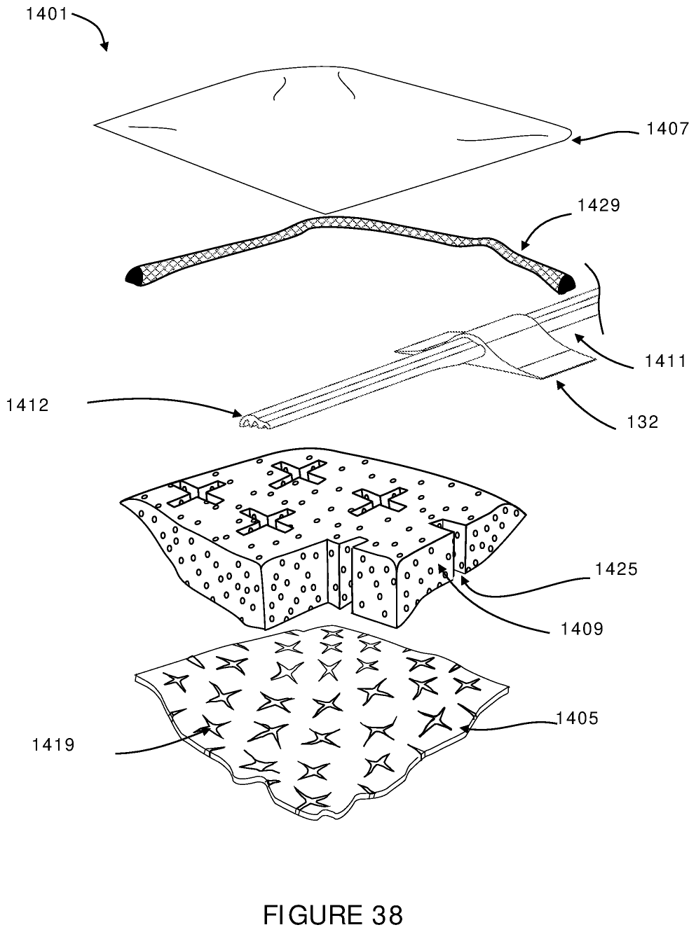

With reference to the embodiment shown in FIG. 37 and FIG. 38, the porting layer 1409 comprises a hydrophilic, non-reticulated polyurethane foam that comprises an absorbent polymer, such as sodium polyacrylate, within the foam sub layer 1423 to provide a hydrophilic property. The hydrophilic non-reticulated polyurethane foam is a medium density foam with a pore size of approximately 200 μm to approximately 400 μm. In other embodiments, the foam contains a distribution of pore sizes ranging from approximately 10 μm to approximately 600 μm. In some forms, the foam may be a dense foam with a pore size of approximately 20 μm to approximately 50 μm, and may contain a distribution of pore sizes ranging from approximately 5 μm to approximately 150 μm.

The surface of the porting layer 1409 may comprise a layer of silicone that interfaces with the bioresorbable layer 1405. In the embodiment of FIGS. 37 and 38, the porting layer 1409 comprises a staggered pattern of x-shaped channels 1425 that are comparable to the shape illustrated in FIG. 16 (i). The channels 1425 are approximately 12 mm in height and approximately 12 mm in width with a slot width of approximately 2 mm when presented to the wound site. The height and width of the channels 1425 may vary from approximately 3 mm to approximately 15 mm and may also vary in width from 0.5 mm to 8 mm. The channels 1425 are spaced in staggered pattern with repeating channels 1425 spaced approximately 20 mm apart along a first axis and approximately 10 mm apart on a second axis which is perpendicular to the first axis. The porting layer 1409 is approximately 8 mm thick, but may otherwise be from approximately 3 mm thick to approximately 30 mm thickness depending on the wound 103 geometry, such as depth, and comfort requirements of the site, such as areas around the lower spine and buttocks.

In another embodiment the porting layer 1409 may comprise a medium density PVA foam with a distribution of pore sizes ranging from approximately 10 μm to approximately 600 μm. The foam may also be a dense foam with a distribution of pore sizes of approximately 20 μm to approximately 30 μm. The PVA foam may also retain an antimicrobial agent. The porting layer 1409 may also contain a series of x-shaped channels 1425 arranged in a staggered pattern. The channels 1425 are approximately 6 mm in height and approximately 6 mm in width with a slot width of approximately 1.5 mm when measured in a non-hydrated or dry format. The height and width of the channels 1425 may vary from approximately 3 mm to approximately 15 mm and may also vary in width from approximately 0.5 mm to approximately 8 mm. The channels 1425 are spaced in staggered pattern with repeating channels 1425 spaced approximately 14 mm apart along a first axis and approximately 7 mm apart on a second axis which is perpendicular to the first axis. The porting layer 1409 is approximately 5 mm in thickness when measured in a dry format but may also be from approximately 2 mm thick to 20 mm thickness depending on the wound 103 geometry, or may be abutted together to treat areas such as deep tunnelling wounds or areas where there is undermining.

The porting layer 109 has wicking properties to wick liquid away from the bioresorbable layer. PVA foams, unlike reticulated foams, are denser as they contain a higher amount of PVA material in the cell walls of the foam pores which allow high levels of moisture to be absorbed and retained within the foam. When utilised for the porting layer 109, the PVA provides a gradient of fluid absorbance which draws excess moisture away from the bioresorbable layer to allow cells important to wound healing to migrate into the bioresorbable layer and proliferate. PVA foams that are combined with antimicrobial agents also additionally reduce the infection risks associated with retained wound fluid sitting on a wound while they can also elute the antimicrobial agent to manage high bioburden levels and unwanted microbial activity. Examples of such antimicrobials can include silver, tetracyclines, gentium violet, methylene blue and chlorhexidine.

In addition to the multiplicity of fluid pathways inherent in the foam, provided for by the porosity of the foam, the porting layer 109 may comprise an array of through channels 125 that are substantially perpendicular to the interface between the porting layer 109 and the bioresorbable layer 105 and extend through the full thickness of the porting layer 109. These channels 125 reduce the pressure drop across the porting layer 109 to ensure negative pressure is effectively applied to the bioresorbable layer 105.

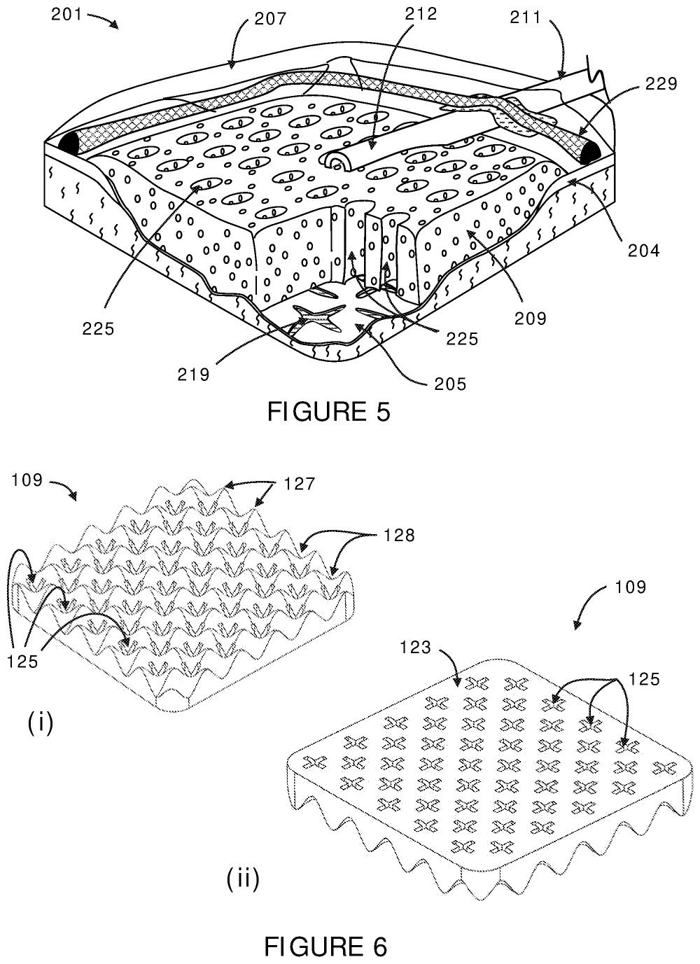

The through channels 125 are preferably linear and x-shaped, as shown in FIG. 37, but may otherwise be of any suitable cross-section and configuration, such as round or oval as shown in the alternative embodiment of FIG. 5. The cross-section of the through channels is preferably many times larger than that of the fluid passages inherent in the foam.

The through channels 124, together with the semi-closed nature of the foam, ensure that a low pressure drop is provided across the porting layer 109 to allow the effective application of negative pressure, but while reducing tissue in-growth. FIG. 12 illustrates the process of tissue growing into a through channel 124 of the porting layer as the wound heals. However, because ingrowth of the tissue into the foam itself is minimal, the porting layer can be lifted from the wound without also removing significant amounts of tissue, as shown in FIG. 12(iv).

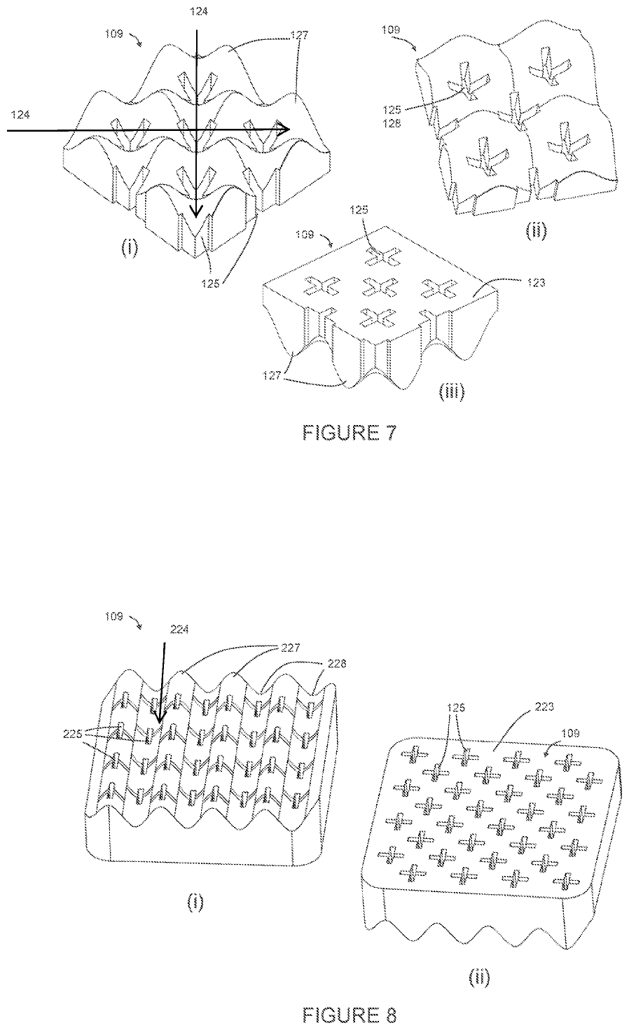

In the embodiment of FIG. 3, an upper surface of the porting layer 109 is undulating with peaks 127 and valleys 128. The occlusive layer 107 is stretched across the porting layer 109, sitting on the peaks 27 such that at the valleys 28, a gap is formed between the occlusive layer and the adjacent top surface of the porting layer 109. Referring to FIG. 7 (i), these gaps form fluid flow paths 124 to assist the application of negative pressure across the whole width of the porting layer 109, and also to assist the passage of fluids from the porting layer 109 to the conduit 111.

Referring to FIGS. 6(i) to 7(iii), in the embodiment of FIG. 3, the upper surface of the porting layer 109 is undulating in two directions with an array of peaks 127 defining a lattice of diagonally intersecting flow paths 124. The openings of the through channels 124 are situated in the valleys 128 between the peaks 127 such that they are in fluid communication with the fluid flow paths passing between the peaks 127.

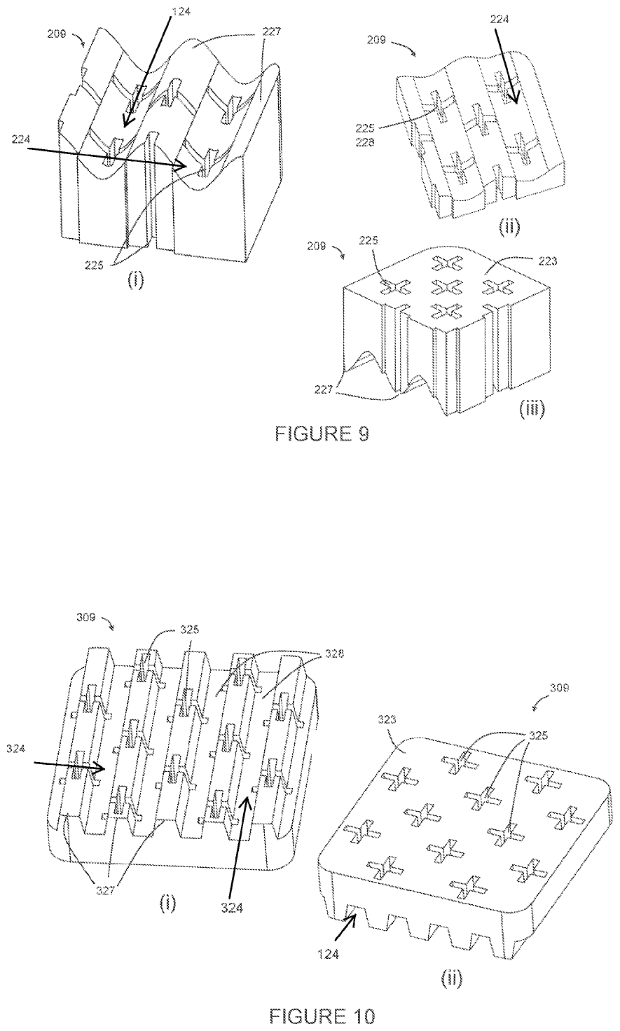

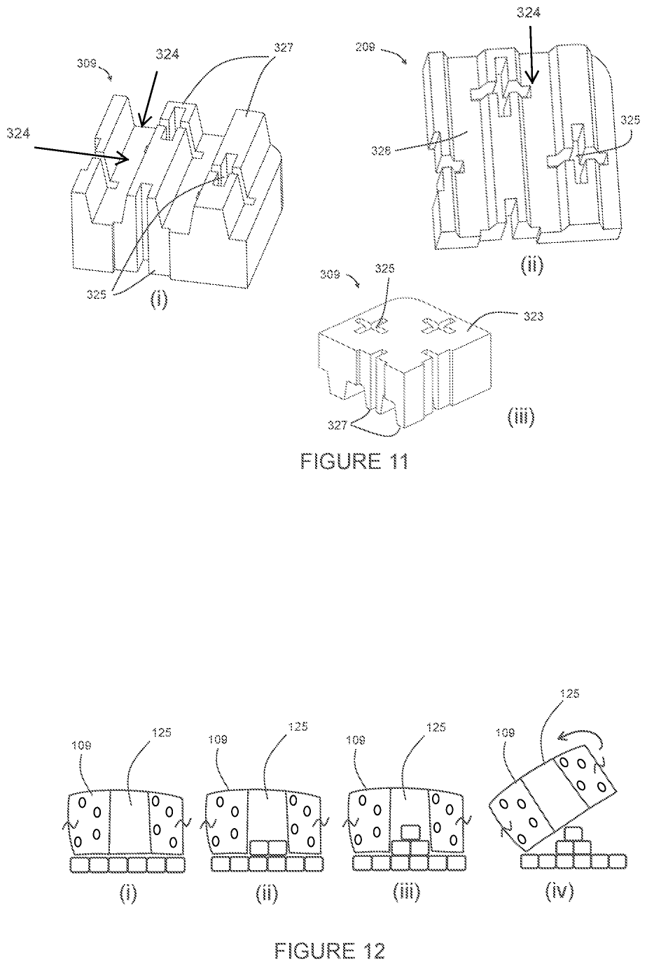

In alternative embodiments, the upper surface of the porting layer 109 may comprise a series of ribs 327. The ribs 327 may be curved as shown in FIGS. 8(i) to 9(iii), or they may be stepped, as shown in the example of FIGS. 10(i) to 11(iii). In the embodiment of FIG. 10(i), the openings of the fluid channels 324 span the peaks of the ribs 327. This allows the free flow of fluids along the adjacent valleys 328, thereby ensuring that the supplied negative pressure is evenly distributed across the porting layer 109. The distributed pressure is then ported via the channels 324 to the underside 323 of the porting layer, to evenly distribute pressure across the adjacent bioresorbable layer.

The porting layer may be either partially or completed adhered to the bioresorbable layer or may have a non-adhesive surface. Additionally, the lug features of the bioresorbable layer could be inserted into the foam to mechanically lug the two components together.

Fluid Conduit

The fluid conduit 111 comprises a flexible tube, for example a plastic or elastomeric walled tube. The tube may have a wall thickness sufficient to avoid the walls collapsing under the applied negative pressure, for example, 50-250 mmHg, or up to 650 mmHg. Suitable conduits utilised for wound therapy purposes will be apparent to those skilled in the art. Alternatively, the conduit 111 may comprise a thin wall supported by a bracing truss or other material or structure to prevent the walls of the tube collapsing under negative pressure. For example, the conduit 111 may comprise a tube comprising a membrane or thin wall surrounding a resilient coil or an open cell foam or three dimensional fabric or matrix.

A first end of the conduit 111 is configured for attachment to a source of negative pressure such as a pump (not shown) or other common negative pressure wound therapy system. For example, the conduit 111 may have an end coupling such as a luer connector or threaded connector for attaching to a negative pressure source. Alternatively, the conduit 111 may be sized to receive or to be received by a suitable connector as would be apparent to a skilled person.

A second end 112 of the conduit 111 is in fluid communication with the porting layer 109 and arranged to apply pressure to the porting layer 109. In the embodiment shown in FIGS. 3 & 4, the second end 112 of the conduit is positioned between the occlusive layer 107 and the porting layer 109. A distal end of the conduit 111 extends into an opening formed between the occlusive layer 107 and the patient. In some forms, the distal end of the conduit 111 may be secured to the patient's skin to reduce the risk of the second/distal end of the conduit being inadvertently pulled from the dressing 101. For example, the distal end of the conduit 111 may be secured using a piece of adhesive tape 131 placed over the conduit 111 and adhered to the skin. In the embodiment shown, the tape is positioned between the edge of the pressure distribution layer 106 and the adjacent edge of the occlusive layer 107 and covered by the occlusive layer 107.

FIGS. 35 (i) and 35 (ii) illustrate one form of sleeve 132 that comprises a passage 137 for receiving a portion of the conduit 111. The sleeve 132 forms a separator that defines a negative pressure region on one side of the sleeve and an ambient pressure region on the other side of the sleeve 132. A hermetic seal can be formed between the exterior surface of the conduit 111 and the interior surface of the passage 137, such as by fastening the conduit 111 to the sleeve 132 by bonding, welding or adhering the conduit to the sleeve. The underside, or skin contacting side, of the sleeve may comprise an adhesive layer 136 such as a medical grade acrylic based pressure-sensitive adhesive, a silicone gel adhesive or other suitable adhesive material to assist with locating and securing the conduit and sleeve to the skin. In some forms, the sleeve comprises an elastomeric material to provide an element of flex and grip.

Alternatively, the occlusive layer 107 may comprise an opening and the second/distal end 112 of the conduit 111 may terminate at the upper surface of the occlusive layer 107 so as to apply negative pressure across the underside of occlusive layer and those layers beneath the occlusive layer, in a similar manner to the arrangement of the dressing shown in FIG. 2. Alternatively, the distal end of the conduit 111 may extend through an aperture in the occlusive layer 107 to apply negative pressure across the layers of the wound dressing beneath the occlusive layer. In some embodiments, the occlusive layer is sealed to the distal end of the conduit to prevent fluid leaks.

The conduit 111 may comprise a dual lumen conduit having a primary lumen 133 to supply negative pressure to the dressing and one or more secondary lumens 134. The secondary lumens 134 may be utilised for introducing fluid to the wound site or to enable measurement and monitoring of the pressure within the dressing. Alternative embodiments may instead comprise a plurality of conduits to introduce fluids to the wound site and to monitor pressure across the site.

In the embodiment shown in FIG. 34, the conduit 111 comprises a primary lumen 133 and a smaller secondary lumen 134 arranged side-by-side. The primary lumen 133 has an elliptical profile with a major axis length of approximately 4.5 mm and minor axis length of approximately 3.8 mm, while the secondary lumen 134 has a circular profile of approximately 1.5 mm in diameter. The open area of the primary lumen 133 is approximately 10 mm² while the open area of secondary lumen is approximately 2 mm². The elliptical profile of this embodiment coupled with the side-by-side arrangement is intended to ensure that the conduit 111 lays generally flat against the various contours of the body in situations where the source of negative pressure is located remotely from the wound, such a situation being likely when the wound is located on the upper arm or lower leg regions of the body.

The dual lumen conduit 111 in this embodiment is preferably made from a medical grade thermoplastic elastomer, with a ‘soft feel’, preferably of a Durometer Hardness of between Shore 30 A and Shore 80 A, to ensure comfort against the skin and wound when pressure is applied to the dressing 101 during use, such as when a patient may be lying on the dressing 101 for long durations.

It is also important that the surface texture of the conduit material has a low coefficient of friction to prevent unwanted bioburden and particulate accumulating on the surface of the conduit, such is the issue with polysiloxane (silicone) materials. However, the conduit could be made from any other readily available elastomeric material such as thermoplastic polyurethane, synthetic rubber, silicone or other plasticized synthetic polymers.

The embodiment in FIG. 34 also incorporates an angled strut 135 along the minor axis of the primary lumen 133 to prevent the collapse of the tube when pressure is applied to the top of the conduit 111 or dressing 101, such as when the patient may be lying on top of device. This strut 135 provides the conduit with a soft and conforming profile to reduce the risk that the conduit may create pressure related injuries to the patient after prolonged localised compression between the patient's body and the conduit, particularly where the conduit is in contact with the spine, hip, ankle, knee or shoulder.

The second/distal end 112 of the conduit or a portion of the conduit adjacent the second end 112 may have an enlarged open area for receiving fluid into the conduit 111 and for better distributing the pressure from the conduit 111 across the porting layer. For example, the conduit may be provided with an elongate elliptical opening, such as by providing a tapered distal end 112. In some forms, the taper of the distal end 112 may be so gradual as to allow the distal end of the conduit to sit almost flat against an upper surface of one of the layers of the wound dressing, such as the occlusive layer 107. This enlarged open area of the distal end reduces the likelihood of the conduit becoming blocked at the distal end 112 and also aids in the distribution of negative pressure across the surface of the porting layer. In alternative embodiments, the conduit may comprise a series of teeth within the internal lumen of the conduit, so that if a portion of the wall of the conduit is cut away to provide a tapered distal end 112, the teeth are exposed and prevent collapse of the remaining portion of the tube wall under compression. This may also reduce the point loading of the conduit onto the wound, which is a limitation of the existing prior art (FIG. 2), and which can cause the conduit port to be pressed into the wound causing pain.

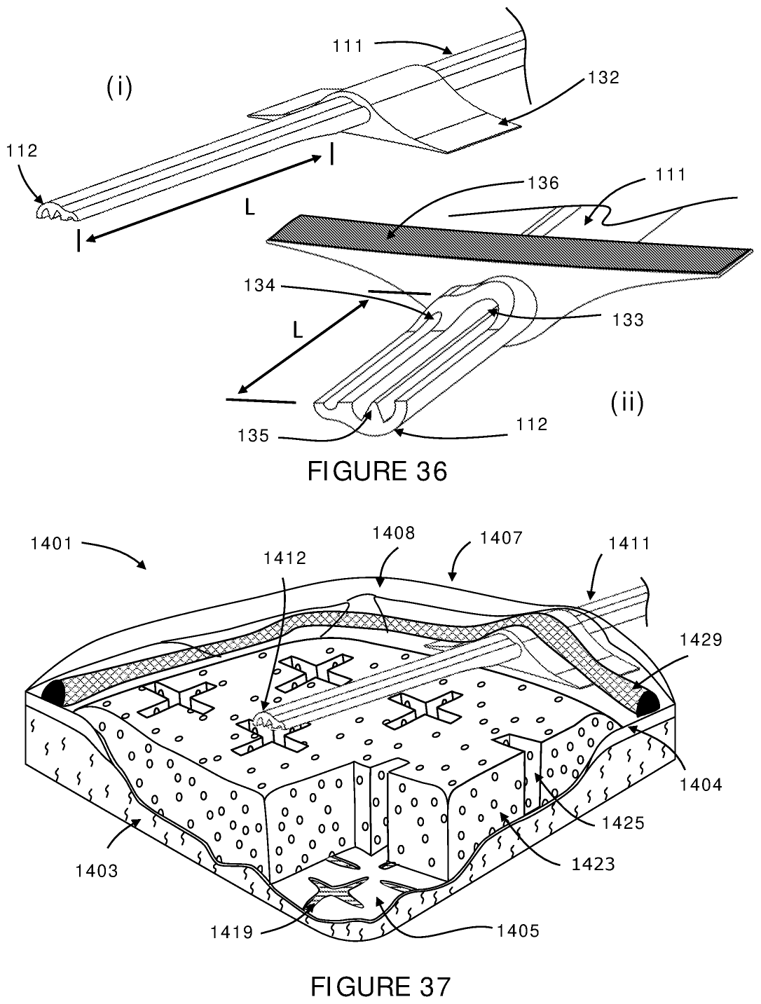

With reference to the alternative embodiment shown in FIGS. 34, 36 (i) and 36 (ii), the conduit 111 comprises a dual lumen conduit where the elongate opening at the distal end 112 has been formed by removing a length of the conduit extending from an area near the elastomer sleeve 132 to the terminal end point of the distal end 112 of the conduit. In effect, the distal end ‘L’ portion of the dual lumen conduit is open along the lower side to expose each of the lumens to the upper surface of the dressing layer below. This arrangement is preferably oriented to preserve the strut 135 of the dual lumen conduit, to help prevent collapse of the remaining portion of the conduit, and to expose the secondary lumen 134 along the same length ‘L’. The exposed interior surface of the distal end portion ‘L’ is intended to be supplied in a sufficient length to extend substantially across the longest axis of the wound and to facilitate the distribution of negative pressure across the uppermost surface of the porting layer. Provided that the dual lumen conduit is sufficiently flexible, the conduit 111 may be positioned in any shape or pattern across the top of the porting layer to ensure adequate distribution of the supplied negative pressure.

In the embodiment shown in FIGS. 3 and 4, the distal end portion of the conduit, adjacent the second/distal end 112, is substantially arch-shaped, with the underside of the arch open to the top of the porting layer 109. In this embodiment, the length of the arched end portion of the conduit is about 30% of the width of the dressing, but in alternative embodiment, the length of the end portion may be between about 20% and about 90% of the width of the dressing 101.

In an alternative embodiment, the second/distal end 112 may split into a plurality of branches that each extend in different directions across the top surface of the porting layer 109 to assist in distributing pressure and receiving fluid into the conduit 111 from across the full area of the porting layer 109. Each of these branches may be of a similar internal diameter to the main conduit 111, or smaller, and may comprise an arched portion with an underside in fluid communication with the porting layer 109.

Pressure Distribution Layer

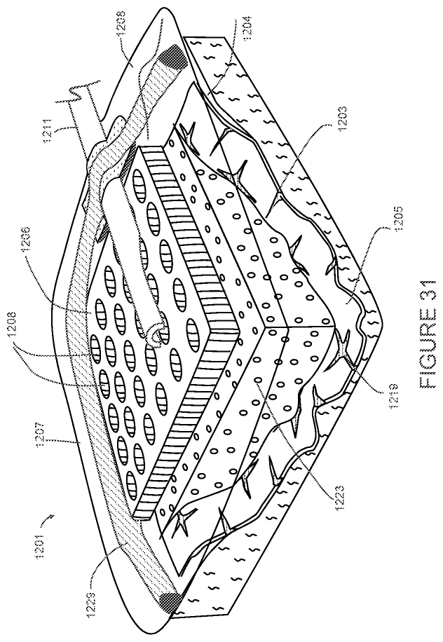

Optionally, the dressing may comprise an additional layer between the porting layer and the occlusive layer. FIGS. 31 to 33 show such alternative embodiment dressings 1201, 1301. The additional layer is a pressure distribution layer 1206 for distributing pressure applied by the conduit 1211 substantially across the full surface of the underlying porting layer 1209, 1309. The pressure distribution layer 1206, 1306 sits directly on the porting layer 1209, 1309, between the porting layer and the occlusive layer 1207, 1307.

The pressure distribution layer 1206, 1306 is flexible and compressible to conform to and cushion the wound site. The pressure distribution layer 1206, 1306 has an open form defining a multiplicity of fluid pathways between the conduit 111 and the porting layer 1209, 1309, to minimise the pressure drop across the thickness of the pressure distribution layer 1206, 1306. As examples, the pressure distribution layer 1206, 1306 may comprise an open cell foam, or a three-dimensional fabric, such as spacer fabric. The openness of the pressure distribution layer 106 is higher than the openness of the material of the porting layer 1209, 1309, the openness at least partly offsetting the pressure drop from using a more closed material such as silicone foam for the porting layer.

In the embodiments shown, the pressure distribution layer 1206, 1306 comprises a three dimensional woven polyethylene fabric. The woven layer has an open form that defines a lattice of pressure distribution channels between the threads forming the fabric. The multi-directional flow paths ensure fluid can always flow through much of the layer even if some pathways become blocked.

Optionally, the pressure distribution layer 1206 may comprise an array of interconnected pressure distribution channels 1208 that are substantially perpendicular to the interface between the porting layer 1209, 1309 and the pressure distribution layer 1206, 1306. Fluid can flow along these vertical channels but also sideways between the channels, that is, between the threads forming the fabric. In alternative embodiments, the pressure distribution layer may comprise an open cell foam, for example reticulated foam.

Occlusive Layer

The occlusive layer 107 is substantially liquid impermeable and substantially air impermeable. Preferably, the occlusive layer 107 has a high water vapour transmission rate (WVTR), also known as Moisture Vapour Transmission Rate (MVTR), to provide a sealed environment for the application of negative pressure but to allow moisture to exchange through the dressing. This helps to prevent maceration of the intact peri-wound and also allows excess fluids and exudate to vent out of the wound environment. An underside of the occlusive layer 107 optionally comprises an adhesive surface for removably adhering a peripheral portion of the dressing 101 to a patient's skin to seal the wound cavity and thereby allow control of the pressure within the cavity.

The surface area of the occlusive layer 107 is preferably larger than that of the underlying bioresorbable and porting layers 105, 109, with the peripheral portion of the occlusive layer 107 optionally forming an adhesion flap 108 for adhering to the peri-wound 104 to secure the dressing in place. In some embodiments, the adhesive coating may only be applied to the underside (patient contacting side) of this adhesion flap 108.

The adhesive surface of the occlusive layer may be created by applying an adhesive coating to all or to a peripheral portion of the underside (patient contacting side) of the occlusive layer 107. Where the adhesive coating is applied to all of the underside of the occlusive layer, the occlusive layer 107 may optionally be adhered to the porting layer 1091.

In another forms, an adhesive or seal may be separately applied around the periphery of the occlusive layer or a sealing layer may be placed over the occlusive layer so s to extend beyond the periphery of the occlusive layer to adhere and seal the wound dressing to the patient's skin.

The occlusive layer 107 may be substantially transparent or may comprise a transparent region to enable monitoring of the underlying layers. In one embodiment, the porting layer 109 comprises one or more viewing apertures that are located beneath the transparent region to enable visual inspection of at least a portion of the bioresorbable layer. This may assist with monitoring the progress of the wound healing.

In the embodiment shown, the occlusive layer 107 is a transparent thin polyurethane based sheet (for example, about 15-60 μm thick, preferably about 20 μm thick to provide good MVTR while still being easy to handle), and having a skin friendly 20-80 μm thick layer of silicone adhesive applied to the underside. Alternative adhesives include modified rubber based adhesives and pressure sensitive acrylic adhesives, or a combination thereof.

Mouldable Seal

To improve the liquid tightness of the seal between the dressing and the patient's skin surface, and to protect the peri-wound area 104, a mouldable seal/adhesive 129 may be placed around the wound perimeter, but preferably within the boundary of the occlusive layer 107. The occlusive layer 107 is typically placed over the mouldable seal/adhesive 129 and adheres to the skin around the outside of the area defined by the mouldable strip 129. In other forms, the mouldable seal may be placed over and around the periphery of the occlusive layer 107 to seal against both the occlusive layer and the patient's skin.

The mouldable seal/adhesive may comprise a non-curing mouldable material. Typically, the mouldable seal comprises a homogenous material in which the adhesive strength is generally consistent throughout the material. This allows the material to be stretched, deformed, kneaded and manipulated to create any shape whilst maintaining a high level of adhesive strength. Consequently, the mouldable seal/adhesive may be repositionable, deformable and stretchable.

In the embodiments shown, the mouldable seal comprises a butyl-rubber based adhesive component. The component comprises synthetically sourced butyl-rubber which has been mixed with a tackifying resin agent, an organic filler to deaden the rubber compound into a soft form tacky singular form, and optionally a stabilising agent. In the preferred embodiment, the compound consists of Polyisobutylene, an aliphatic hydrocarbon resin as a tackifying agent, calcium carbonate as a filler material and Poly(dicyclopentadiene-co-p-cresol) as a stabilising agent. Alternatively, any suitable hypoallergenic tackifying resin could be used during the mixing and extrusion process of making the seal material, while other filler materials could include talc, dolomite, barytes, kaolin and silica. In alternative embodiments, the mouldable seal may comprise alternative mouldable adhesives or alternative rubber sources, such as mouldable polysiloxane (silicone), styrene butadiene, polychloroprene (neoprene), nitrile rubber or compounds that include blends of the aforementioned synthetic rubbers.

The mouldable seal/adhesive 129 offers several other advantages such as high levels of skin adhesion with a low level of trauma or pain during removal. The adhesive properties of the mouldable seal can be adjusted by varying the amount of tackifier added during the mixing and extrusion process, which can be adjusted to achieve comparable adhesion properties to the acrylic based pressure sensitive adhesives typically used for medical dressings and devices to achieve high skin adhesion. Unlike coated adhesive dressings, the mouldable seal/adhesive 129 can be stretched off the skin after use so as to break adhesion between the adhesive surface of the seal and the skin. The mouldable seal/adhesive 129 is also removable and repositionable on the skin while retaining a high level of adhesive strength.

A further advantage of the mouldable seal/adhesive 129 is the ability to directly apply release agents comprising either isopropyl alcohol (IPA), hexamethyldisiloxane, 1,1,1,2-tetrafluoretan, ISOPARAFFIN L, (2-methoxymethylethoxy) propanol, Hydrotreated heavy naphtha (petroleum) or a blend of agents to the mouldable seal/adhesive 129 as required during the removal of the dressing.

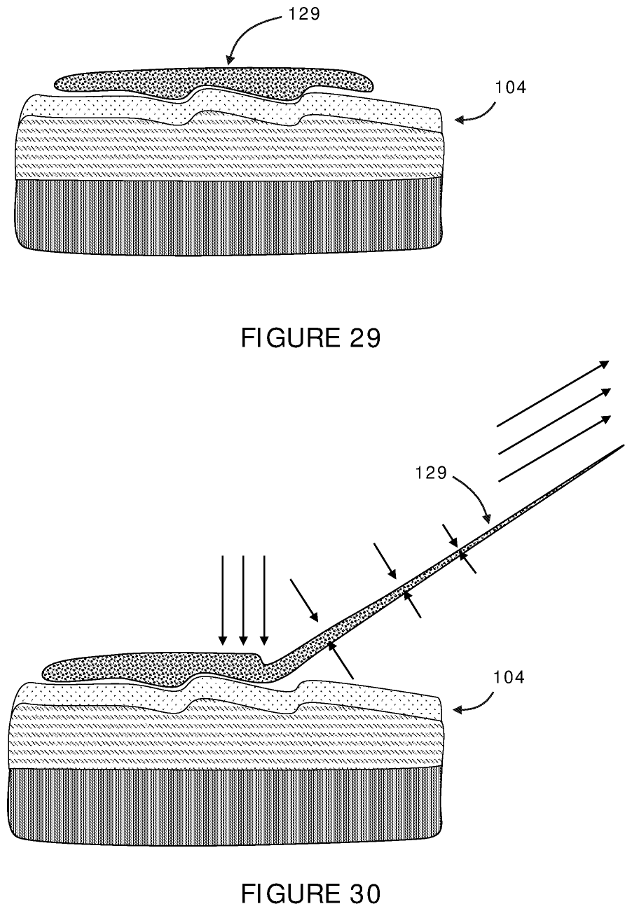

Other advantages of the mouldable seal/adhesive 129 include its thickness and softness which allows the material of the seal to be depressed and moulded into skin folds and crevices that are common on patients and which can lead to leaks and subsequent loss of negative pressure to the wound, as shown in FIG. 29, if an ineffective seal is formed between the dressing and skin. The softness of the mouldable seal/adhesive can also be adjusted by changing the rubber material used during the mixing and extrusion process. The amount of filler added during manufacture can increase or decrease the softness of the mouldable seal/adhesive.

The mouldable seal 129 may be provided in a strip form. In some forms, the seal is provided in a strip comprising a width of approximately 10 mm, a thickness of approximately 3 mm and a length of approximately 250 mm. In some forms, the seal strip may otherwise be provided in any widths ranging from approximately 5 mm to approximately 30 mm and with a thicknesses ranging from approximately 2 mm to approximately 8 mm, and a length ranging from approximately 50 mm to approximately 400 mm. In some forms, the mouldable seal 129 may be provided in a roll with a total length ranging from approximately 200 mm to approximately 5000 mm. In some forms, the seal may be manually formed to a desired shape from a block of mouldable material, such as by shaping the material into an elongate, long sausage-shaped strip.

In some forms, an elongate, flat strip of the mouldable material is provided on a first removable release sheet, which is adhered to one side of the mouldable strip 129. A second removable release sheet is adhered to the opposite second side of the mouldable strip, such that the mouldable strip is sandwiched between the releasable sheets. In other forms the mouldable adhesive 129 is provided in a roll with release sheets attached to both sides of the overlapping surfaces of the roll.

The first removable release sheet may be a paper-based material or any other suitable material, such as a plastic material, that is attached to a first surface of the mouldable strip, i.e. the patient-contacting side of the mouldable strip. This first removable release sheet protects the mouldable strip during storage and handling and is removable to expose a first surface of the mouldable seal strip. The surface of the paper-based material contacting the mouldable strip may be coated with a release coating, such as silicone or any other release agents such as Polytetrafluoroethylene (PTFE), to reduce the adhesion between the first removable release sheet and the mouldable strip for easy removal of the first release sheet.

The second removable release sheet adheres to a second surface of the mouldable strip opposite the first surface. This second removable release sheet protects the mouldable strip during placement of the strip around the wound. The second removable release sheet is preferably a thin flexible sheet of silicone or any other suitable material that is able to stretch along with the mouldable strip 129 to allow the mouldable strip to be manipulated and shaped as required to conform to the peri-wound site, without removal of the second release sheet. The surface of the second release sheet contacting the mouldable strip may be coated with a release coating to reduce the adhesion between the second removable sheet and the mouldable strip for easy removal of the second release sheet.

The second release sheet is preferably transparent or semi-transparent such that the skin surface the mouldable strip is being applied to is visible to the clinician during application in order to assist with the application. For example, the second removable release sheet may comprise a silicone sheet.

The second removable release sheet is removed from the mouldable strip after the mouldable strip is applied to the patient, thereby removing the need for a medical professional to touch the surface of the mouldable strip to apply it.

A removable protector sheet is adhered to the second removable release sheet to protect the second removable release sheet during transport. This protector sheet may be a paper-based material or may be of any other suitable material, such as a plastic material that is removable to expose the second release sheet.

Application and Removal of the Dressing

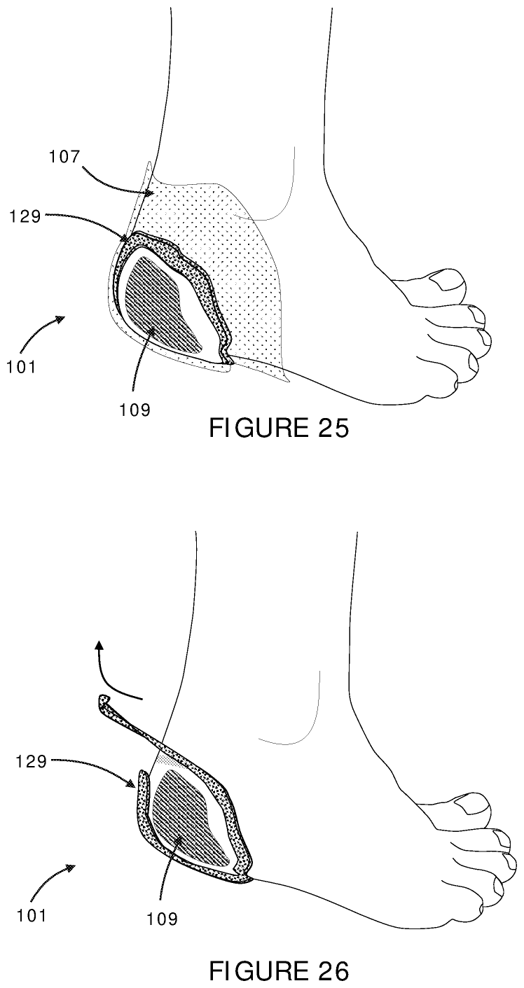

The dressings 101, 201, 401 described above and other embodiments thereof are intended for use in the treatment of chronic wounds, for example diabetic ulcers and burns. FIGS. 25 and 26 illustrate the dressing 101 applied to a foot wound and an arm wound respectively.

To apply the dressing, the mouldable material is first applied around the peri-wound as described above. The mouldable material is pressed into the skin 104 surrounding the wound, thereby filling in skin undulations and creases as illustrated in FIG. 29, to act as a skin barrier and reduce fluid leakage from the dressing.

The bioresorbable layer 105 is then fitted over the wound surface in full contact with the surface and following the wound contours. The porting layer 109 is then placed over the bioresorbable layer, the conduit 111 is secured in place over the porting layer 109, and the arrangement is then covered by the occlusive layer and sealed to ensure the wound is air-tight.

The first end of the conduit 111 is coupled to a source of negative pressure such as a vacuum pump, and the pump operated to create a continuous or intermittent vacuum within the sealed dressing. The negative pressure assists the removal of fluid from the wound and may improve circulation to improve wound healing.

To remove the dressing, the occlusive layer 107 is peeled off the skin, and the conduit 111 and porting layer 109 are removed. The vacuum pump may comprise a reservoir for collecting exudate liquids removed from the dressing.

Referring to FIGS. 26, 28, and 30, in a final step, the mouldable seal 129 can be removed from the patient's skin by stretching the mouldable seal in a longitudinal direction as illustrated. As the seal is elongated, it gently releases from the patient's skin 104 minimising the likelihood of damage to the skin.

The bioresorbable layer does not need to be removed from the wound, as it breaks down naturally with time.

### EXAMPLES

### Example 1: Method of Manufacture

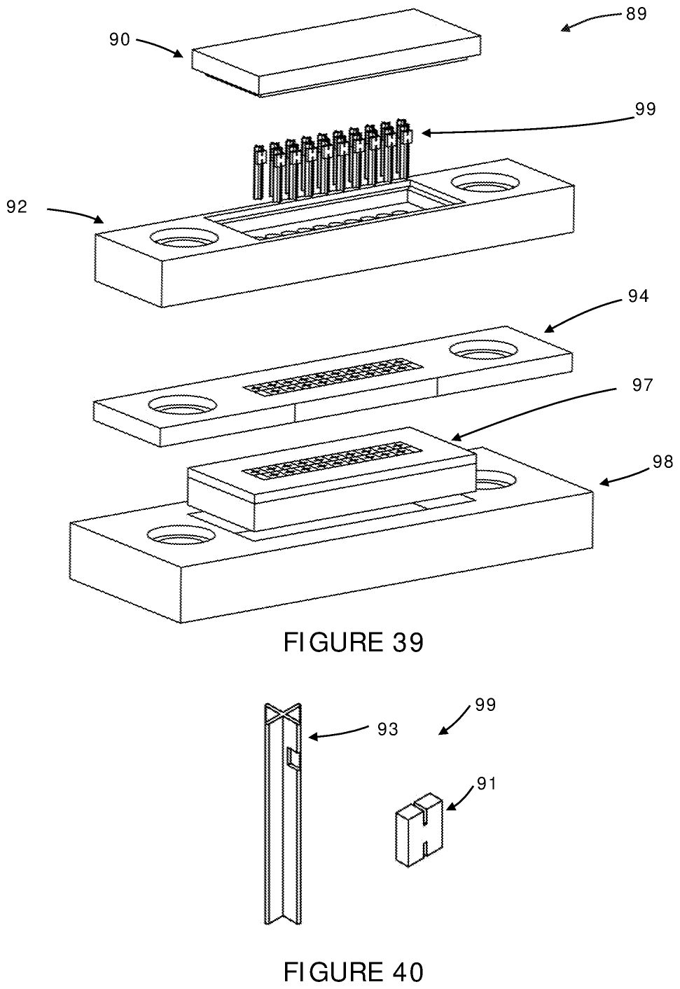

A bioresorbable layer 105 with x-shaped apertures 119 was prepared according the following method. With reference to FIGS. 39-41, a sheet of reticulum tissue was prepared according to the method described within PCT application PCT/NZ2009/000152 to provide a material 95 for processing using a blanking/perforating apparatus 89.

The blanking apparatus 89 includes an upper press assembly that comprises a punch retainer plate 92, a punch pin assembly 99 and a clamping plate 90. The punch pin assembly 99 includes a punch 93 that is shaped to produce the desired final aperture, and a punch pin retainer 91.

In this example, the punch 93 is shaped to produce the x-shaped aperture 119 shown in FIG. 16 (i) and has a width and height of approximately 5.5 mm with a slot thickness or width of approximately 0.5 mm.

The blanking apparatus 89 includes a presser plate 94, which is aligned to traverse along the same axis as the upper press assembly, defined by the punch retainer plate 92, punch pin assembly 99 and a clamping plate 90, and the lower die plate 97, which is retained by a die nest 98.

The punch retainer plate 92, presser plate 94 and lower die plate 97 are aligned with high precision to ensure a tight clearance fit with the punch 93 when it travels through the presser plate 94 and lower die plate 97 during the blanking/perforating process.

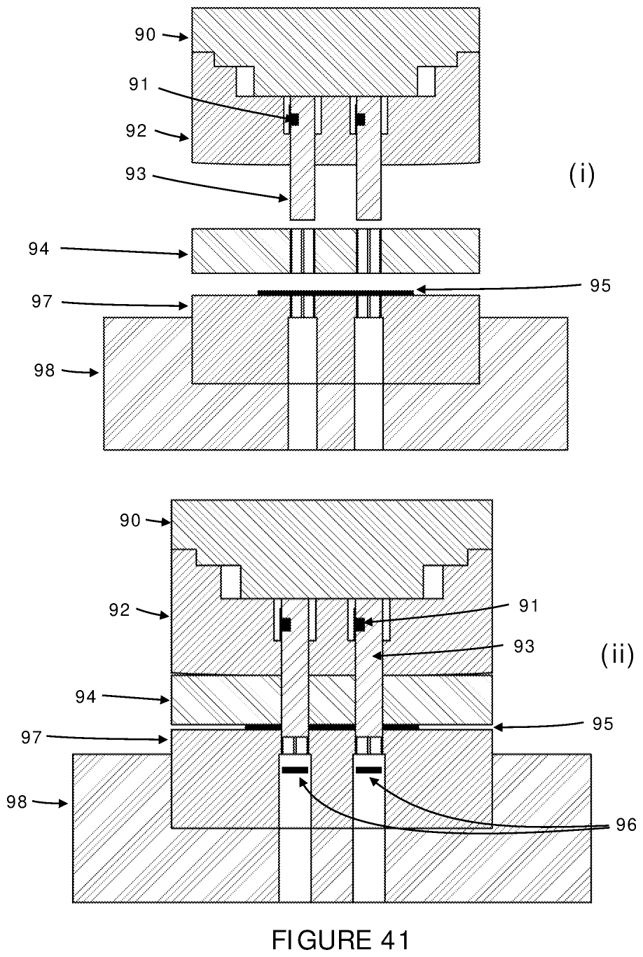

With reference to FIG. 41(i), the material for blanking 95 is placed on the lower die plate 97 and the pressure plate 94 and upper press assembly is contracted to advance the punch 93. The presser plate 94 engages with the material 95 to firmly hold the sheet in place while the upper press assembly continues to advance.

FIG. 41(ii) illustrates the punch 93 passing through the material 95 to blank/punch out a slug 96 of material, thereby forming the aperture 119 in the material 95. In other words, the apertures are formed in the layer of bioresorbable material, by removing a slug from the layer of material. The material 95 is then indexed, or moved in a transverse direction, to repeat the process until the required number of apertures 119 are formed in the material.

### Example 2: Measuring Pressure Drop Across Various Bioresorbable Layers

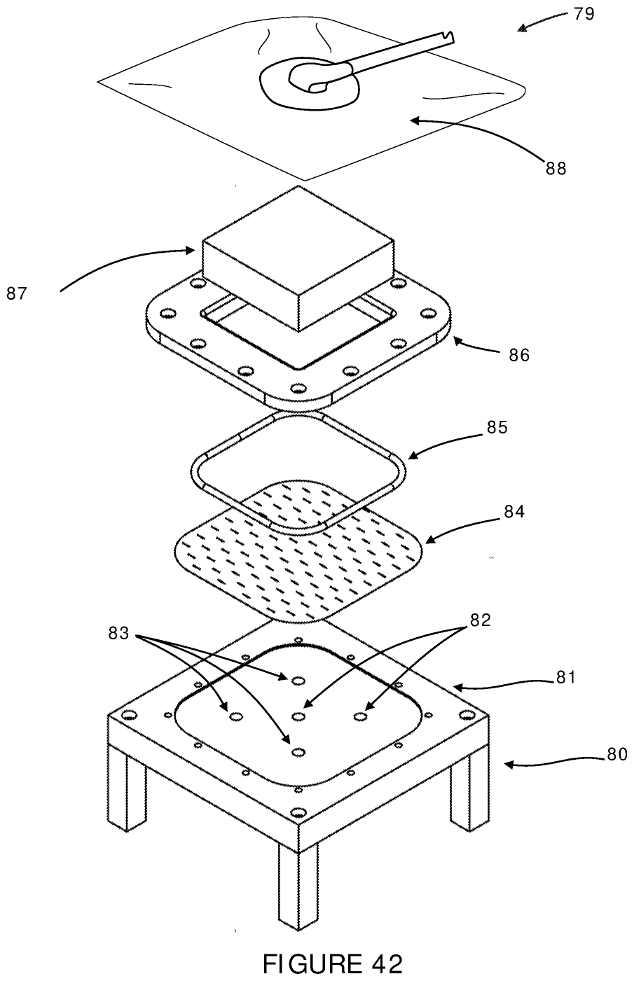

The following example outlines the apparatus and test method used to assess and compare the pressure drop across various materials and different forms of bioresorbable layers. With reference to FIG. 42, a pressure drop test apparatus 79 was constructed that comprises a base plate 81, base support 80 and clamping ring 86. The clamping ring 86 includes a peripheral groove (not shown in FIG. 42) to house a rubber o-ring or other form of seal 85.

The base plate 81 includes two ports 82 that are each connected to a separate pressure sensor. Each pressure sensor is capable of accurately measuring vacuum pressure across a range of 0-400 mmHg. The pressure sensors are referred to herein as measurement points P2 and P3. The two ports 82 are spaced approximately 45 mm apart. The base plate 81 may also include three spare ports 83 that are spaced approximately 45 mm away from a central pressure measuring port 82.

A test specimen 84 was cut to fit within a central recess on the base plate 81 and was subsequently secured in place by fastening the clamping ring 86 to the base plate 81 using the fastening components located around the periphery of the clamping ring 86 and the base plate 81.

Prior to proceeding further, the specimen was rehydrated within the test apparatus according to the manufacturer's instructions and excess fluid was removed from the apparatus. For the materials that relate to the described invention, test specimens were rehydrated using physiological saline for approximately 5 minutes and any excess fluid was removed from the apparatus prior to testing.

All testing was performed by placing a 100 mm×100 mm sized piece of reticulated open-cell polyurethane foam 87 (V.A.C.® GRANUFOAM®—K.C.I/Acelity®) in the central opening of the clamping ring 86, which was sized to ensure a leak-free and consistent fit with the foam. Each test was then performed with an adhesive polyurethane drape (V.A.C.® Drape—K.C.I/Acelity®) and centrally placed portal 87 (SENSAT.R.A.C.™ Pad—K.C.I/Acelity®) affixed over the top of the setup.

A ‘y-connector’ was then fitted to the centrally placed portal to allow one end of the conduit to be connected to a pressure sensor with the other end of the conduit to be connected to a controlled source of negative pressure. The pressure sensor placed next to the drape is herein referred to as measurement point P1. Prior to and following any testing the pressure sensors at measurement points P1, P2 and P3 were verified to ensure the calibration was within specification.

Each test was then performed according to the following sequence:
1. Pressurise measurement point P1 to the required pressure level.
2. Maintain pressure for 5 minutes to verify the system is free of any leaks.
3. Maintain the pressure set point for a further 5 minutes while logging the pressure from measurement points P1, P2 & P3 at 1 second intervals.
4. Repeat the test cycle 3 or more times for the same test specimen to establish the average pressure measurements at the 3 measurement points (P1, P2 & P3).
5. Assess the data to determine whether P1 was held constant within ±5 mmHg throughout the 5 minute data acquisition period (step 3 above).
6. Remove the test specimen and perform two further replicate tests of the same test specimen to determine the pressure drop of each material type.

The test was performed with four different materials where a ‘Foam Only’ test was performed as a control. The four different materials were denoted as the following:
1. BI-LAYER CGAG—A bioengineered composite wound matrix that comprises Type-1 Bovine collagen that has been cross linked with glycosaminoglycan. This wound matrix is affixed to a layer of silicone and been fenestrated using a series of staggered linear cuts, similar to that of FIG. 18.
2. CORC—A resorbable composite collagen dressing that comprises 45% oxidized regenerated cellulose (ORC) and 55% collagen.
3. 3-PLY LUGGED—A 3-ply multi-layer bioresorbable layer similar to that of FIG. 14 (i), prepared according to the method described PCT application PCT/NZ2015/050215, using lyophilized sheets of ovine forestomach matrix, or rumen sourced and processed according to PCT application PCT/NZ2009/000152.
4. 3-Ply LUGGED with APERTURE—A 3-ply multi-layer bioresorbable layer, the same as 3 above, where the layer comprised x-shaped apertures prepared according to example 1 with an aperture geometry of 5 mm long×5 mm wide with a slot thickness or width of approximately 0.5 mm, where the apertures were positioned in a staggered pattern with repeating apertures positioned 20 mm apart on a first axis and 10 mm apart on a second axis that lies perpendicular to the first axis.

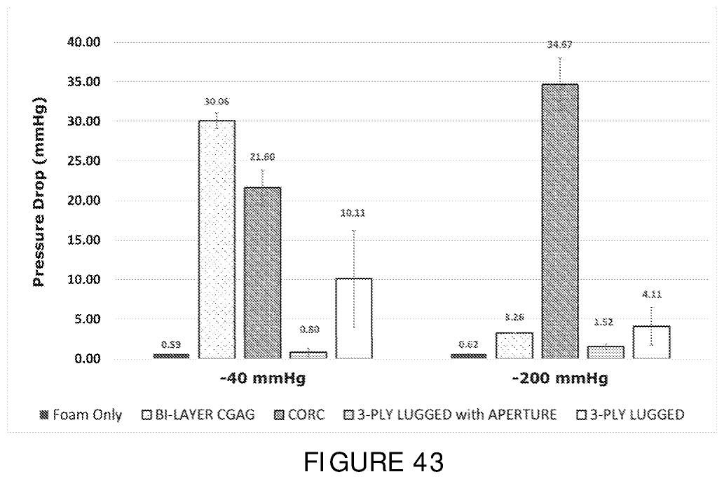

The testing was performed at a vacuum pressure of 40 mmHg and 200 mmHg with the results from the testing shown in FIG. 43.

1. A wound dressing for applying negative pressure to a wound, the dressing comprising:
a bioresorbable layer, for placement in contact with the wound;
a liquid impermeable occlusive outer layer;
a fluid porous porting layer positioned between the outer layer and the bioresorbable layer; and
a fluid conduit in fluid communication with the porting layer, for coupling to a source of negative pressure;
wherein the porting layer comprises a multiplicity of fluid pathways between the conduit and the bioresorbable layer;
and wherein the bioresorbable layer comprises a plurality of apertures to enable fluid flow from the wound to the porting layer.

2. A wound dressing as claimed in claim 1, wherein the bioresorbable layer comprises a plurality of mechanically interlocked bioresorbable sheets.

3. A wound dressing as claimed in claim 1 or claim 2, wherein the bioresorbable layer comprises a first sheet having a plurality of lugs and a second sheet having a plurality of apertures, each lug of the first sheet being located through a respective aperture in the second sheet to interlock the first sheet with the second sheet.

4. A wound dressing as claimed in any one of the preceding claims, wherein the bioresorbable sheets comprise extracellular matrix (ECM).

5. A wound dressing as claimed claim 4, wherein the ECM comprises reticulum.

6. A wound dressing as claimed in any one of the preceding claims, wherein the bioresorbable layer comprises a plurality of apertures, the apertures defining fluid pathways.

7. A wound dressing as claimed in claim 6, wherein the apertures comprise two intersecting slots to form a cross shape and to define one or more flaps in the bioresorbable layer, wherein the flaps are movable to increase the size of the opening provided by each aperture.

8. A wound dressing as claimed in claim 7, wherein the apertures are substantially X-shaped, Y-shaped, C-shaped, U-shaped, or V-shaped.

9. A wound dressing as claimed in any one of claims 6 to 8, wherein the apertures are formed through the bioresorbable layer by removing a slug of material from the bioresorbable layer.

10. A wound dressing as claimed in any one of claims 1 to 5, wherein the bioresorbable layer comprises a plurality of slits defining said fluid pathways, each slit defining one or more flaps in the bioresorbable layer, wherein the flaps are movable to increase the size of the opening provided by the slit.

11. A wound dressing as claimed in claim 10, wherein the slits are substantially X-shaped, Y-shaped, C-shaped, U-shaped, or V-shaped.

12. A wound dressing as claimed in any one of claims 5 to 11, wherein the slits or apertures are die cut from the bioresorbable layer.

13. A wound dressing as claimed in any one of the preceding claims, wherein the porting layer comprises a fluid-permeable foam.

14. A wound dressing as claimed in claim 13, wherein the porting layer comprises PVA foam.

15. A wound dressing as claimed in any one of the preceding claims, wherein the porting layer is compliant and porous.

16. A wound dressing as claimed in any one of the preceding claims, wherein an upper surface of the porting layer is undulating.

17. A wound dressing as claimed in claim 13 or claim 14, further comprising a pressure distribution layer between the porting layer and the occlusive layer.

18. A wound dressing as claimed in claim 17, wherein the pressure distribution layer comprises an open cell foam or a three-dimensional fabric.

19. A wound dressing as claimed in claim 17 or claim 18, wherein the pressure distribution layer comprises a plurality of fluid flow channels that are substantially perpendicular to the interface between the foam layer and the pressure distribution layer to allow fluid to flow through the pressure distribution layer.

20. A wound dressing as claimed in any one of the preceding claims, wherein the conduit comprises a distal end portion having an opening in fluid communication with the porting layer.

21. A wound dressing as claimed in claim 20, wherein the distal end portion is substantially arch-shaped.

22. A wound dressing as claimed in claim 20, wherein the conduit comprises a dual lumen conduit comprising a strut positioned along a central axis of one of the lumens to prevent the conduit from collapsing under compression.

23. A wound dressing as claimed in claim 22, wherein the conduit comprises a lumen which is elliptical in shape.

24. A wound dressing as claimed in any one of the preceding claims, wherein the conduit is a dual lumen conduit comprising a primary conduit to apply a negative pressure to the dressing and a secondary conduit for introducing fluid to the dressing or for facilitating pressure measurement.

25. A wound dressing as claimed in any one of the preceding claims, wherein the dressing further comprises a sleeve comprising a port for receiving a portion of the conduit therein in a secure arrangement to attach the conduit to the dressing.

26. A wound dressing as claimed in claim 25, wherein the sleeve comprises an elastomeric material.

27. A wound dressing as claimed in claim 25 or claim 26, wherein the sleeve forms a divider between a negative pressure receiving area of the dressing and an ambient pressure area.

28. A wound dressing as claimed in any one of the preceding claims, wherein the occlusive layer comprises a substantially transparent region and the porting layer comprises one or more viewing apertures to enable visual inspection of at least a portion of the bioresorbable layer.

29. A wound dressing as claimed in any one of the preceding claims, wherein the occlusive layer comprises a polyurethane sheet comprising an adhesive surface.

30. A wound dressing as claimed in any one of the preceding claims, wherein the wound dressing comprises a mouldable adhesive seal for surrounding a wound, wherein the seal comprises butyl rubber, a filler, and a tackifying resin.

31. A wound dressing as claimed in claim 30, wherein the seal is removable and re-sealable against a patient's skin.

32. A wound dressing as claimed in claim 30 or claim 31, wherein the seal is non-curing.

33. A wound dressing as claimed in any one of claim 30 to claim 32, wherein the seal is removable from a skin surface by stretching the adhered seal.

34. A mouldable and removable adhesive seal for surrounding a wound, the seal comprising butyl rubber, a filler, and a tackifying resin.

35. A mouldable adhesive seal application system comprising the mouldable adhesive seal of claim 34, and further comprising a first removable release sheet adhered to one side of the adhesive seal, and a second removable release sheet adhered to a second side of the adhesive seal, wherein the second removable release sheet is stretchable.

36. The adhesive seal application system as claimed in claim 35, wherein the second removable release sheet comprises silicone.

37. The adhesive seal application system as claimed in claim 35 or claim 36, wherein the first removable release sheet is paper based and comprises an adhesive contacting side coated in silicone.

38. The adhesive seal application system as claimed in any one of claims 35 to 37, further comprising a removable protector sheet adhered to the second removable release sheet.

39. The adhesive seal application system as claimed in any one of claims 35 to 38, wherein the adhesive seal is elongate and stretchable.

40. The adhesive seal application system as claimed in any one of claims 35 to 39, wherein the seal is non-curing.

41. A wound treatment system comprising a wound dressing as claimed in any one of claims 1 to 33, and the mouldable adhesive seal of claim 34, wherein the mouldable adhesive seal is applied around the perimeter of the wound to the patient's skin.

42. A wound treatment system as claimed in claim 41, wherein the occlusive layer is adhered over the mouldable adhesive seal.

43. A wound treatment system comprising a wound dressing as claimed in any one of claims 1 to 33, wherein a negative pressure source is coupled to the conduit to apply a negative pressure to the wound.

44. A wound treatment system as claimed in any one of claims 41 to 43 and further comprising a reservoir for collecting exudate removed from the dressing.
